(19) United States
(12) Patent Application Publication
(10) Pub. No.: US 2024/0367156 A1
(43) Pub. Date: Nov. 7, 2024

(54) IRON-COBALT-BASED MAGNETICALLY RECOVERABLE CATALYSTS AND METHOD OF PREPARATION THEREOF

(71) Applicant: KING FAHD UNIVERSITY OF PETROLEUM AND MINERALS, Dhahran (SA)

(72) Inventor: M. Nasiruzzaman SHAIKH, Dhahran (SA)

(73) Assignee: KING FAHD UNIVERSITY OF PETROLEUM AND MINERALS, Dhahran (SA)

(21) Appl. No.: 18/312,998

(22) Filed: May 5, 2023

(51) Int. Cl. B01J 23/75; B01J 21/18; B01J 35/00; B01J 35/02; B01J 37/00; B01J 37/08; C07D 307/44

(52) U.S. Cl. CPC B01J 23/75; B01J 21/18; B01J 35/23; B01J 35/33; B01J 35/393; B01J 35/50; B01J 37/0036; B01J 37/086; C07D 307/44

(57) ABSTRACT

A magnetically recoverable catalyst that includes a core material and a shell material, where the core material is an Fe—Co alloy containing Fe and Co in an equal molar ratio and the shell material is a graphitic carbon. A process for producing the magnetically recoverable catalyst includes mixing of nanoparticles of Fe3O4 with a Co metal-organic-framework material. The magnetically recoverable catalyst of the present disclosure can be used for selective hydrogenation of furfural.

### STATEMENT OF PRIOR DISCLOSURE BE INVENTOR

Aspects of the present disclosure are described in M. Nasiruzzaman Shaikh, Muhammad Ali, Mahmoud M. Abdelnaby, Abbas S. Hakeem, Mohammed A Sanhoob, Huda S. Alghamdi, Afnan Ajeebi, and Md. Abdul Aziz; “Facile Hydrogenation of Furfural by MOF-Derived Graphitic Carbon Wrapped FeCo Bimetallic Catalysts,” Chemistry: An Asian Journal; Apr. 7, 2023; 202201254; incorporated herein by reference.

### STATEMENT OF ACKNOWLEDGEMENT

Support provided by King Fahd University of Petroleum & Minerals (KFUPM) under grant number INHE2212 is gratefully acknowledged.

### BACKGROUND

### Technical Field

The present disclosure is directed to catalysts, and particularly to iron-cobalt-based magnetically recoverable catalysts and the method of preparation thereof.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

The transition from a petroleum-based chemical ecosystem to a system based on bio-derived renewable resources, especially non-edible lignocellulose, has drawn considerable attention to address the crises of depleting fossil-fuel reserves and environmental issues. Furfural, derived from the hydrolysis of lignocellulose, is a crucial platform molecule containing an aldehyde group (—C═O) and a conjugated double bond system (—C═C—C═C—) used to produce liquid hydrocarbon fuels or fuel additives and synthesize value-added chemicals. For instance, furfuryl alcohol (FAL), obtained by hydrogenating the carbonyl group of furfurals, is widely used for producing lubricants, resins, plasticizers, ascorbic acids, and fibers. The reduction of both functional groups, i.e., the carbonyl group and the furan ring, produces tetrahydro furfuryl alcohol (TFAL), which is transparent, miscible with water, high boiling, and a green solvent used in agricultural applications, printer inks, and electronic cleaners. Moreover, furanic ethers are used additives with excellent blending properties without adverse effects on engine performance, a low CO₂ emission footprint, and a high-octane number.

Currently, two existing routes for furfuryl ethers preparation are available: (1) furfural hydrogenation to furfuryl alcohol (FAL) and then acid-catalyzed etherification of alcohols; and (2) the direct reductive etherification by the reaction of an aldehyde with alcohol in a hydrogen environment in one step. Methods of furfural hydrogenation followed by acid-catalyzed etherification are known in the art. An H form of Zeolite Socony Mobil-5 (HZSM-5), which is a zeolite-based catalyst capable of both enhancing hydrogenation and providing Bronsted acidity for etherification, demonstrated 80% conversion of furfural to ethyl furfuryl ether (EFE) was reported. An Amberlyst-15-catalyzed selective etherification of furanyl alcohols by both ethanol and butanol has been reported [Sacia, E. R.; Balakrishnan, M.; Bell, A. T. Biomass Conversion to Diesel via the Etherification of Furanyl Alcohols Catalyzed by Amberlyst-15. J. Catal. 2014, 313, 70-79]. A ZSM-5-based catalyst for the synthesis of furfuryl ether in the presence of trimethyl orthoformate or triethyl orthoformate at very low temperatures has also been reported.

However, only a very few reports of efficient methods for the synthesis of furfural ether in a single step have been published. A palladium/carbon (Pd/C)-catalyzed single-step etherification of furfural with a 77% selectivity for methyl furfuryl ether (MFE) has been reported. Recently, a one-pot synthesis of furfuryl ether with an 83% selectivity using supported Pd nanoparticles was reported. Reductive etherification involves a shorter route and a lower reaction temperature, hindering the decomposition of the produced ether compared to the hydrogenation-etherification process. However, studies on the efficiency of a catalyst in terms of quantitative conversion in a single step and exclusive selectivity towards a given furfuryl ether among the wide variety of products are rare.

Hence, an efficient and robust single-step synthesis procedure based on a non-noble-metal-based catalyst, which may be conveniently separated from the reaction medium and recycled for multiple cycles, is highly desirable. In addition, LA is identified as an automobile fuel extender and an important building block of methyltetrahydrofuran (MTHF), a miscible oxygenate with favorable vapor pressure properties. For a long time, LA has been synthesized by the hydrolysis of acetyl succinate esters, oxidation of ketones with ozone, and Pd-catalyzed carbonylation of ketones. However, these processes have yet to find commercial-scale applications due to the high cost of feedstock, complicated production processes, and the lack of product selectivity. Thus, a commercially viable and environmentally benign process to produce this important chemical is highly desirable.

Catalysis has primarily relied on rare transition metals for decades at a high cost. However, using base transition metals offers several advantages other than cost-multiple spin states, high-density states, and geometric and electronic structure can open new reaction pathways and overcome issues associated with the substrate commonly encountered with using precious metals. Moreover, alloying metal components have been found to enhance the activity and selectivity further owing to the additional degree of freedom for modifying the geometric and electronic effects. Such interaction enables a catalyst based on an alloy to achieve superior hydrogenation activity and selectivity over its monometallic counterpart. Iron and cobalt are two such environmentally friendly and of lower cost metals. Combining these two metals in their nano-sized forms (Fe—Co nanoparticles) captured the advantages of the spin states and accessible oxidations with new mechanistic pathways, unique substrate scope, or altogether new reactivity.

Published literature indicates a growing interest in using metal-organic-frameworks (MOFs) in catalysis. MOFs possess abundant diversity, flexibility, a rich structural diversity between the metal nodes, a wide variety of organic linkers, space for sheltering guest metal nanoparticles without disruption of the framework, and selective heterogeneous interaction. The use of MOFs in carbon capture, hydrogen storage, sensing, membranes, and catalysis has been reported.

Superparamagnetic iron oxide nanoparticles (SPIONs) are known for their distinct magnetic nature with the advantages of being the low cost of raw materials, negligible toxicity, high stability, and ease of surface functionalization. Furthermore, their high resistivity to oxidation in air and moisture, relative stability, and the formation of extremely small (<5 nm) Fe₃O₄ particles in a well-controlled MOF architecture may lead to the development of new materials and a system with enhanced physicochemical properties.

Although the promise of the bimetallic system is undeniable, harnessing its full potential is somewhat diminished due to certain shortcomings. The bare surface of these Fe—Co bimetallic nanoparticles is prone to oxidation, and they have low thermal stability; and therefore the anticipated catalytic activity may not be realized. However, covering the edges with a surface-protecting agent prevents aggregation and oxidation. Moreover, some of these surface agents even enhance the selectivity of important chemical processes by influencing the substrate's adsorption geometries on the catalyst's surface. Hence, there is a requirement for the judicious choice of a surface-protecting agent in the bimetallic system.

Accordingly, it is one object of the present disclosure to provide magnetically recoverable catalyst material comprising an FeCo alloy core and a graphitic carbon shell. It is also the object of the present disclosure to provide a method for hydrogenating furfural into furfural alcohol or furfuryl ether using a magnetically recoverable catalyst material comprising an FeCo alloy core and a graphitic carbon shell

### SUMMARY

In an exemplary embodiment, a magnetically recoverable catalyst is described. The magnetically recoverable catalyst includes a core material and a shell material, where the core material is an iron-cobalt (Fe—Co) alloy containing Fe and Co in an equal molar ratio and the shell material is a graphitic carbon.

In some embodiments, the magnetically recoverable catalyst has the Fe—Co core which is 3 to 4 nanometers (nm) in diameter.

In some embodiments, the magnetically recoverable catalyst has a particle diameter ranging from 20 to 250 nm.

In some embodiments, the magnetically recoverable catalyst has a particle diameter of about 40 nm.

In some embodiments, the core material has a body-centered cubic (BCC) crystal structure.

In some embodiments, the core material has a Raman D-band to a G-band ratio (I_{D}/I_{G}) between 0.50 and 0.80 at the D-band equal to 1350-1370 and the G-band equal to 1575-1595 inverse centimeters (cm⁻¹).

In another exemplary embodiment, a process for producing the magnetically recoverable catalyst is described. The method includes mixing nanoparticles of ferric oxide (Fe₃O₄) with a Co metal-organic-framework (MOF) material and grinding to form a precursor mixture, where the Fe and the Co are present in the precursor mixture in an equal molar ratio and heating to at least 600 degrees centigrade (° C.) under an inert gas.

In some embodiments, the Fe₃O₄ nanoparticles have a diameter ranging from 20 to 250 nm.

In some embodiments, the magnetic oxide particles have a diameter of about 40 nm.

In some embodiments, the Co MOF material is metal-organic framework (MOF)-71 (Co).

In some embodiments, the mixing includes dispersing the Fe₃O₄ nanoparticles having a diameter less than 5 nm in the Co MOF material.

In some embodiments, the process for producing the magnetically recoverable catalyst includes heating to a temperature of 600 to less than 900° C.

In some embodiments, the process for producing the magnetically recoverable catalyst includes heating to a temperature of 600 to about 800° C.

In yet another exemplary embodiment, a process for selective hydrogenation of furfural is described. The process includes combining, in a reactor, the furfural with a catalyst material containing the Fe—Co alloy core and the graphitic carbon shell, where the Fe and Co are present in equal molar ratios. Further, the process includes adding an alcohol, flushing the reactor with at least one time with hydrogen gas, and further pressurizing the reactor to a pressure of at least 10 bars with hydrogen gas and heating the reactor to a temperature of at least 140° C. to hydrogenate the furfural in the reactor in the presence of the catalyst material.

In some embodiments, the process further includes heating the reactor to a temperature from 140° C. to 200° C.

In some embodiments, the reactor is heated to about 170° C.

In some embodiments, the reactor is pressurized to 40 bars during the heating.

In some embodiments, pressurizing, and heating which is conducted for about 20 hours (hrs.).

In some embodiments, the alcohol is a branched alcohol.

In some embodiments, the alcohol is a straight-chain alcohol.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 shows a catalytic conversion of furfural to fuel components and other value-added chemicals;

FIG. 2 is a schematic flow diagram of a process for producing a magnetically recoverable catalyst (iron-cobalt encapsulated with a graphitic carbon (FeCo@GC)), according to certain embodiments;

FIG. 3 is a schematic flow diagram of a process for selective hydrogenation of the furfural, according to certain embodiments;

FIG. 4 shows a synthesis protocol of the magnetically recoverable catalyst, according to certain embodiments;

FIG. 5A shows an X-ray diffraction (XRD) pattern of the FeCo@GC catalyst, at different pyrolysis temperatures, and Fe:Co ratios, according to certain embodiments;

FIG. 5B shows Raman spectra of the FeCo@GC catalyst at different pyrolysis temperatures and Fe:Co ratios, according to certain embodiments;

FIG. 6A shows a field emission scanning electron microscopy (FESEM) of the FeCo@GC (1:1) catalyst at 600° C., according to certain embodiments;

FIG. 6B shows a field emission scanning electron microscopy-energy dispersive spectroscopy (FESEM-EDS) of Fe and Co, according to certain embodiments;

FIG. 6C shows a FESEM image of the FeCo@GC (2:1) catalyst at 600° C., according to certain embodiments;

FIG. 6D shows a FESEM image of the FeCo@GC (3:1) catalyst at 600° C., according to certain embodiments;

FIG. 6E shows a FESEM image of the FeCo@GC (1:1) catalyst at 800° C., according to certain embodiments;

FIG. 6F shows a FESEM image of the FeCo@GC (1:1) catalyst at 900° C., according to certain embodiments;

FIG. 6G shows a FESEM-EDS of Fe in the FeCo@GC (1:1) catalyst at 900° C., according to certain embodiments;

FIG. 6H shows a FESEM-EDS of Co in the FeCo@GC (1:1) catalyst at 900° C., according to certain embodiments;

FIG. 6I shows a transmission electron microscope (TEM) image of iron oxide (Fe₃O₄), according to certain embodiments;

FIGS. 6J-6L show TEM images of FeCo@GC@600 (1:1) catalyst, according to certain embodiments;

FIG. 6M shows a high-resolution transmission electron microscopy (HRTEM) of the FeCo@GC@600 (1:1) catalyst, according to certain embodiments;

FIG. 6N shows a selected area of electron diffraction (SAED) of the FeCo@GC@600 (1:1) catalyst, according to certain embodiments;

FIGS. 6O-6P shows a scanning transmission electron microscopy-high-angle annular dark-field (STEM-HAADF) of the FeCo@GC@600 (1:1) catalyst, according to certain embodiments;

FIG. 7 shows a scanning electron microscopy with energy dispersive X-ray spectroscopy (SEM-EDX) image of elements in the FeCo@GC catalyst, according to certain embodiments;

FIG. 8A shows an XRD plot depicting the investigation of the mechanism of formation of FeCo@GC using temperature-programmed desorption (TPD) reduction, according to certain embodiments;

FIG. 8B shows an XRD plot depicting the mechanism of formation of the FeCo@GC catalyst with Fe₃O₄ and cobalt oxide (Co₃O₄) at 600° C., according to certain embodiments;

FIG. 8C shows an XRD plot depicting the mechanism of formation of the FeCo@GC catalyst with Fe₃O₄ and pristine Co at 600° C., according to certain embodiments;

FIG. 8D shows an XRD plot depicting the mechanism of formation of the FeCo@GC catalyst with Fe₃O₄, pristine Co, and 1,4-benzenedicarboxylic acid (BDC) linker at 600° C., according to certain embodiments;

FIG. 9 shows the XRD data of pure metal-organic framework (MOF)-71, according to certain embodiments;

FIG. 10 shows a thermogravimetric analysis (TGA) in the air for the FeCo@GC catalyst at 600° C., in different ratios, according to certain embodiments;

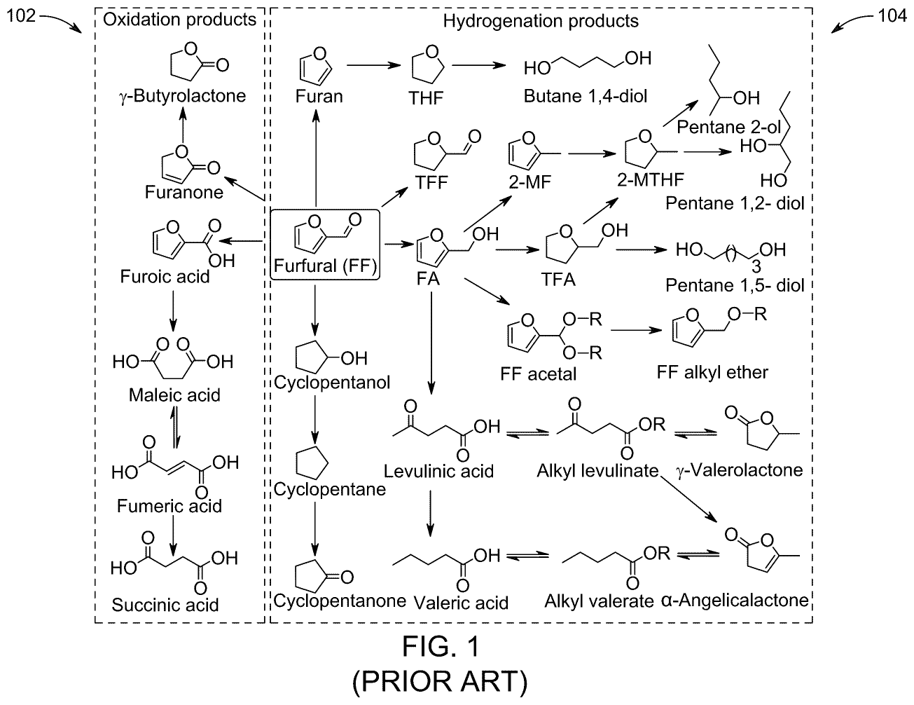

FIG. 1 11A and FIG. 11B show an energy dispersive X-ray-High-angle annular dark-field (EDX-HAADF) image of elements in FeCo@GC by, according to certain embodiments;

FIG. 12A shows an X-ray photoelectron spectroscopy (XPS) global survey spectra of the FeCo@GC (1:1) catalyst, according to certain embodiments;

FIG. 12B shows XPS spectra of cobalt in the FeCo@GC (1:1) catalyst, according to certain embodiments;

FIG. 12C shows an XPS spectra of iron in the FeCo@GC (1:1) catalyst, according to certain embodiments;

FIG. 12D shows an XPS spectra of carbon in the FeCo@GC (1:1) catalyst, according to certain embodiments;

FIG. 13A is a plot depicting the effect of various reaction conditions on the hydrogenation of furfural, according to certain embodiments;

FIG. 13B is a plot depicting the percentage conversion of furfural in isopropanol at 140 and 170° C. with varying H2 pressure, according to certain embodiments;

FIG. 13C is a plot depicting the selectivity of the magnetically recoverable catalyst in the hydrogenation of furfural in isopropanol at 140 and 170° C. with varying H2 pressure, according to certain embodiments;

FIG. 14A is a plot depicting the percentage conversion of furfural in ethanol at 140 and 170° C. with varying H2 pressure, according to certain embodiments;

FIG. 14B is a plot depicting the selectivity of the magnetically recoverable catalyst in the hydrogenation of furfural in ethanol at 140 and 170° C. with varying H2 pressure, according to certain embodiments;

FIG. 14C is a plot depicting the effect of pressure on the formation of levulinate in anhydrous ethanol at 140° C., according to certain embodiments;

FIG. 15 is a plot depicting room temperature hysteresis loops of FeCo@GC nanoparticles with a different composition at different pyrolysis temperatures, according to certain embodiments;

FIG. 16A is a plot depicting the percentage conversion of furfural in butanol at 140 and 170° C. with varying H2 pressure with the magnetically recoverable catalyst, according to certain embodiments;

FIG. 16B is a plot depicting the selectivity of the magnetically recoverable catalyst in the hydrogenation of furfural in butanol at 140 and 170° C. with varying H2 pressure, according to certain embodiments;

FIG. 17 shows a schematic representation of the hydrogenation reaction of furfural, according to certain embodiments;

FIG. 18 shows a mechanism of the furfural hydrogenation to the furfuryl alcohol (FA) over the FeCo@GC catalyst based on density functional theory (DFT) calculations, according to certain embodiments;

FIG. 19A shows a charge redistribution between Fe and Co in a Fe—Co (110) complex, according to certain embodiments;

FIG. 19B shows an electron density contour map of the Fe—Co (110) complex, according to certain embodiments;

FIG. 20A shows the adsorption of H on Fe before geometry optimization of the Fe—Co (110) complex, according to certain embodiments;

FIG. 20B shows H bridges Co atoms after geometry optimization of the Fe—Co (110) complex, according to certain embodiments;

FIG. 21 shows re-usability of FeCo@GC in isopropyl alcohol at 170° C. under 40 bar H2, according to certain embodiments;

FIG. 22A shows gas chromatography (GC) and a mass spectrometry (MS) of the furfural hydrogenation using the FeCo@GC catalyst in isopropyl alcohol at 170° C. under 40 bar H2, according to certain embodiments;

FIG. 22B shows MS spectra of the furfural hydrogenation using the FeCo@GC catalyst in isopropyl alcohol at 170° C. under 40 bar H2, according to certain embodiments;

FIG. 23A shows GC and MS spectra of the furfural hydrogenation using the FeCo@GC catalyst in isopropyl alcohol at 140° C. under 30 bar H2, according to certain embodiments;

FIG. 23B shows GC and MS spectra of furfural during the furfural hydrogenation using the FeCo@GC catalyst in isopropyl alcohol at 140° C. under 30 bar H2, according to certain embodiments;

FIG. 23C shows GC and MS spectra of furfural alcohol during the furfural hydrogenation using the FeCo@GC catalyst in isopropyl alcohol at 140° C. under 30 bar H₂, according to certain embodiments;

FIG. 23D shows GC and MS spectra of Isopropyl furfuryl ether during the furfural hydrogenation using the FeCo@GC catalyst in isopropyl alcohol at 140° C. under 30 bar H₂, according to certain embodiments; and

FIG. 23E shows GC and MS spectra of diether (acetal) during the furfural hydrogenation using the FeCo@GC catalyst in isopropyl alcohol at 140° C. under 30 bar H₂, according to certain embodiments.

### DETAILED DESCRIPTION

In the drawings, reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Additionally, the terms “MOF-71”, “MOF-71 (Co)” and “(MOF)-71 (Co)” are used interchangeably to describe a metal-organic-framework precursor comprising cobalt.

One aspect of the present disclosure is directed to an iron-cobalt (Fe—Co) based magnetically recoverable catalyst. A series of magnetically recoverable iron-cobalt particles encapsulated with a graphitic carbon (FeCo@GC) nano-alloys having a diameter of less than 250 nanometers (nm) are derived from a metal-organic framework (MOF)-71 (Co), (as the Co and carbon (C) source) and iron oxide (Fe₃O₄) nanoparticles.

Another aspect of the present disclosure relates to hydrogenated synthesis of a series of furfuryl ethers and alkyl levulinate from furfural. The furfural is hydrogenated to produce >99% isopropyl furfuryl ether in isopropanol with >99% conversion, while n-chain alcohol, such as ethanol, produces corresponding 93% ethyl levulinate.

In an embodiment of the present disclosure, the magnetically recoverable catalyst comprises a core material. The core material comprises an Fe—Co alloy having a mole ratio (Fe:Co) ranging from 1:1 to 3:1 In some embodiments, the Fe—Co alloy has Fe and Co in an equal molar ratio, preferably 1:1. The Fe—Co core has a diameter ranging from 1 to 10 nanometers (nm), preferably 2 to 7 nm, and more preferably 3 to 5 nm. The magnetically recoverable catalyst has a particle diameter ranging from 20 to 250 nm, preferably about 30-200 nm, and more preferably about 40 nm. The core material has a body-centered cubic (BCC) crystal structure. The core material has a Raman D-band to a G-band intensity ratio (I_{D}/I_{G}) between 0.50 and 0.80 with the D-band between 1350-1370 and the G-band between 1575-1595 inverse centimeters (cm⁻¹).

The magnetically recoverable catalyst also comprises a shell material. Several materials can be used as shell materials, such as mesoporous silica, metal oxides, and other porous materials. In some embodiments, mesoporous silica may be used as the shell material. In certain other embodiments a metal oxide may be used as the shell material. In the preferred embodiment, the shell material is graphitic carbon.

FIG. 1 outlines the important chemicals derived from the furfural. Among them is the important chemical levulinic acid (LA), recognized as a compound associated with the renewable platform derived from lignocellulosic biomass. The oxidation products 102 and the hydrogenation products 104 of the furfural alcohol are shown in FIG. 1.

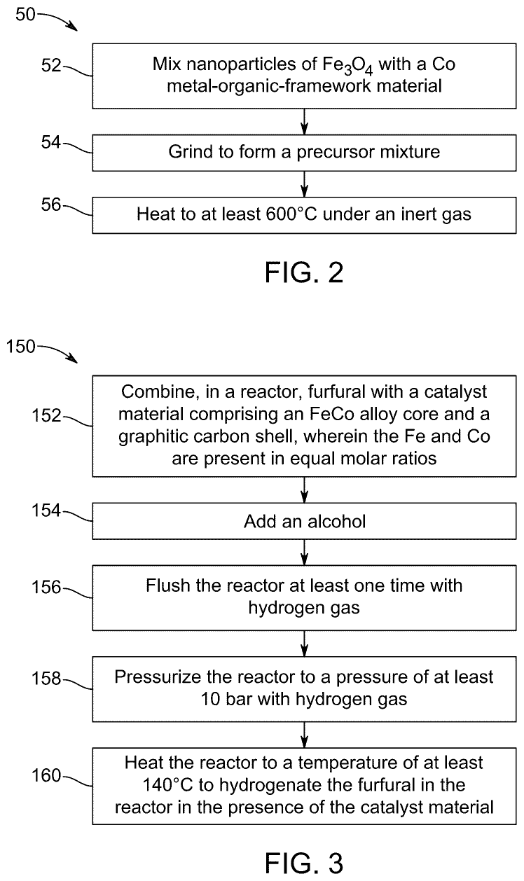

Referring to FIG. 2, a schematic flow diagram of a method 50 a process for producing a magnetically recoverable catalyst is illustrated, according to an embodiment. At step 52, the method 50 includes mixing nanoparticles of Fe₃O₄ with a Co metal-organic-framework material. Magnetic nanoparticles (Fe₃O₄) with a diameter of 3-4 nm were prepared by the method reported by M. N. Shaikh et al. [Shaikh, M. N.; Bououdina, M.; Jimoh, A. A.; Aziz, M. A.; Helal, A.; Hakeem, A. S.; Yamani, Z. H.; Kim, T.-J. The Rhodium Complex of Bis(Diphenylphosphinomethyl)Dopamine-Coated Magnetic Nanoparticles as an Efficient and Reusable Catalyst for Hydroformylation of Olefins. New J. Chem. 2015, 39 (9)].

The metal-organic framework used is generally not limited so long as the chosen metal organic framework is capable of incorporating cobalt. In one embodiment ZIF-67 (Co) may be used as the metal-organic-framework material. In a preferred embodiment, the metal-organic framework is Co-MOF-71.

The weight ratio of Fe₃O₄ and Co-MOF-71 is in a range of 1:1 to 1:2, preferably 1:1. The mixing is carried out in the presence of a nitrogen, or any other carrier gas, for about 1-5 hours, preferably 2-4 hours, more preferably for about 3 hours. Fe—Co nanoparticle with diameter ranging from 37-40 nm with an atomic ratio of 1:1 of Fe to Co were obtained.

At step 54, the method 50 a precursor mixture is formed by grinding the Fe₃O₄ nanoparticles and the Co metal-organic-framework material. Fe and Co are preferably present in the precursor mixture in an equal molar ratio. The grinding is carried out for 10-60 minutes, preferably for 20-40 minutes, more preferably for 30 minutes, to obtain the precursor mixture. The grinding may be carried out with a mortar and pestle, or the grinding may be carried out by mechanical processes.

At step 56, the method 100 includes heating to at least 600° C. under an inert gas. The resulting precursor mixture from step 54 was pyrolyzed, under a continuous flow of nitrogen (10 milliliters per minute (mL/min)) gas, in a tubular furnace, with a ramp rate of 1-10° C./min, preferably 2-8° C./min, preferably 3-7° C./min, preferably 4-6° C./min, preferably at 5° C./min and holding the temperature at 600-800° C., preferably 600° C. for 1-5 hours, preferably 2-4 hours, more preferably 3 hours, to obtain the magnetically recoverable catalyst.

Referring to FIG. 3, a schematic flow diagram of a process 150 for the selective hydrogenation of furfural is illustrated. At step 152, the process 150 includes combining, in a reactor, furfural with a catalyst material including a Fe—Co alloy core and the graphitic carbon shell. The reactor is at least one selected from the group consisting of a fixed-bed reactor, a trickle-bed reactor, a moving bed reactor, a rotating bed reactor, a fluidized bed reactor, and a slurry reactor. In an embodiment, the reactor includes a Teflon-lined autoclave fitted with a pressure gauge and overhead mechanical stirring.

At step 154, the process 150 includes adding an alcohol. The alcohol may be a linear chain or a branched chain. In some embodiments, the alcohol is preferably polar. Suitable examples include ethanol, isopropanol, butanol, or any other polar alcohol. In some embodiments, the alcohol is isopropanol.

At step 156, the process 150 includes flushing the reactor at least once with hydrogen gas. The hydrogen gas is introduced into the reactor to carry out the hydrogenation reaction. The FeCo@GC catalyst was placed inside the reactor and flushed with H₂ three times for hydrogenation.

At step 158, the process 150 includes pressurizing the reactor, preferably to a pressure of at least 10 bars with hydrogen gas. In some embodiments, the pressure applied was about 10-50 bars, preferably 20-40 bars, and more preferably about 40 bars H₂.

At step 160, the process 150 includes heating the reactor to a temperature of at least 140° C. to hydrogenate the furfural in the reactor in the presence of the catalyst material. The reactor is heated to a temperature range of 140-200° C., preferably to about 170° C. with continuous stirring preferably at about 200-10 rpms, preferably 300-800 rpm, and more preferably to about 500 for the hydrogenation of furfural.

### Catalyst Preparation and Characterization

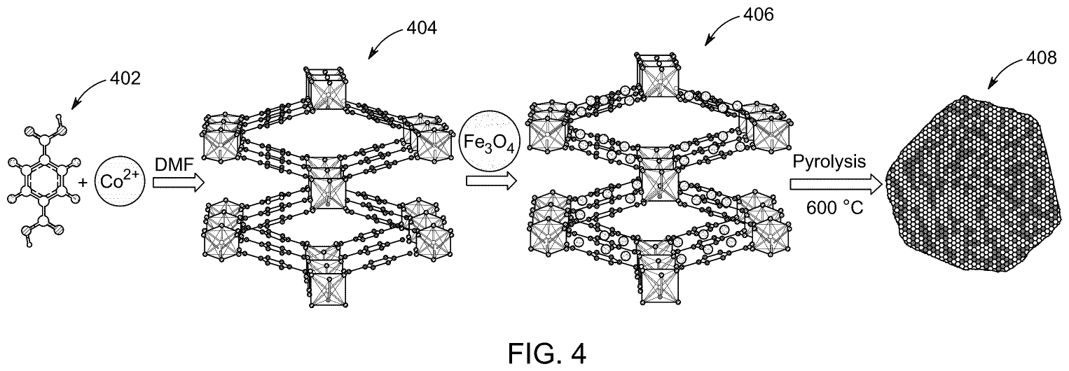

The steps involved in the synthesis of FeCo@GC are shown in FIG. 4. A series of FeCo@GC catalysts were prepared at different temperatures using MOF-71 404 as both the Co and C precursor. In one example, a Fe—Co nanoparticle (37-40 nm) with an atomic ratio of 1:1 of Fe to Co was obtained by feeding an initial weight ratio of 350:650 of Fe₃O₄ and MOF-71 406 under a nitrogen atmosphere for 3 hours at 600° C. In another example, an inhomogeneous particle size distribution was observed at 600° C. when the iron content was increased stepwise from 1:1 to 3:1 (Fe:Co).

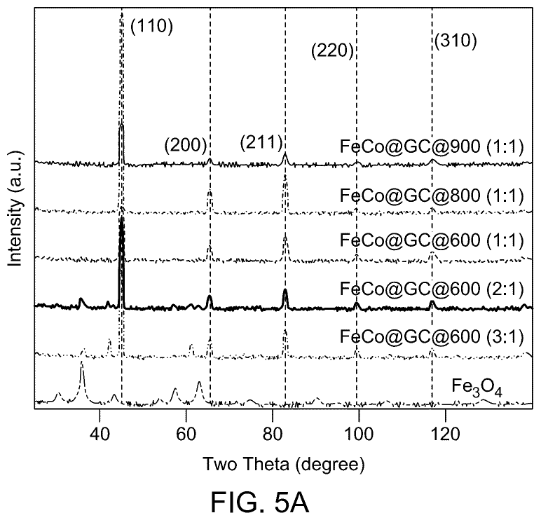

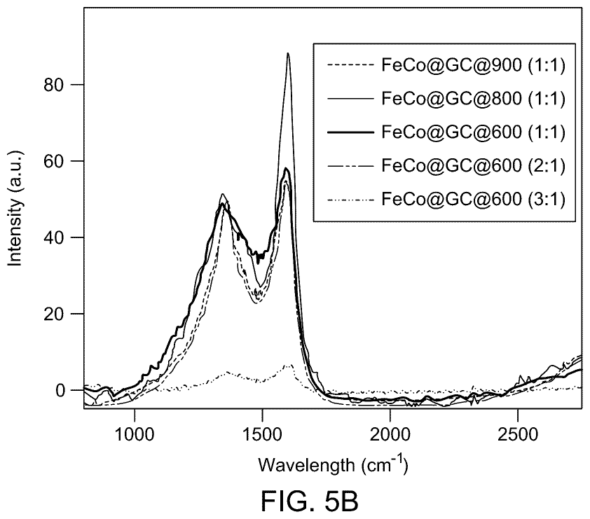

FIG. 5A and FIG. 5B show the effect of pyrolysis temperature and Fe:Co ratios on the FeCo@GC catalyst. The Fe—Co crystallinity was sharply improved at higher pyrolysis temperature 408 (800° C. with an atomic ratio of 1:1), as demonstrated by XRD in FIG. 5A and in Raman spectra (FIG. 5B). FIG. 5A compares the diffraction patterns of the series of FeCo@GC catalysts and pure Fe₃O₄. The signature XRD pattern of the pure phase of Fe₃O₄ in FIG. 5A, reveals peaks at 2θ=29.6, 34.5, 42, 52.5, 56.5, and 73.7° assigned to (220), (311), (400), (422), (511), (440), and (533) planes with inverse spinel (fcc) structure falling under Fd3m space group, respectively. The characteristic diffractions at 2θ=44.8, 65.2, 82.4, and 99.3° of FeCo@GC with varying Co content are ascribed to the (110), (200) (211), and (220) planes of Fe—Co nano-alloy, respectively (JCPDS data with No.: 49-1567). The XRD patterns in FIG. 5A indicates a body-centered cubic (bcc) structure for the Fe—Co core.

Raman spectra (FIG. 5B) have a disordered carbon vibration at 1350-1370 cm⁻¹ (D-band), and 1575-1595 cm⁻¹ band (G-band) due to the in-plane vibration of sp² bonded graphitic carbon structure. The degree of defect level of carbon matrices was quantified as the intensity ratio D-band and G-band (I_{D}/I_{G}). The I_{D}/I_{G} values of FeCo@GC with a composition of 50:50@600, 50:50@800, 50:50@900, 66:33, and 75:25 was 0.75, 0.48, 0.43, 0.49, and 0.59, respectively, indicating the impact of pyrolysis temperature in converting the disordered domain into an ordered graphitic structure at a higher temperature. Carbon with a structure containing higher defects in 50:50@600 exhibited higher catalytic activity. The ordered organic framework (1,4-BDC) around the metal most likely produced carbon with more defects than the random presence of the linker in the mixture. This higher order of defects and pore structure of carbon contributed to improving the stability of the Fe—Co nano-alloy and interacting with metals to enhance the catalytic performance synergistically. Moreover, the stability of FeCo@GC (Fe:Co=1:1) was demonstrated by handling the catalysts for a month in the air without any changes in the Raman spectra and the diffraction patterns.

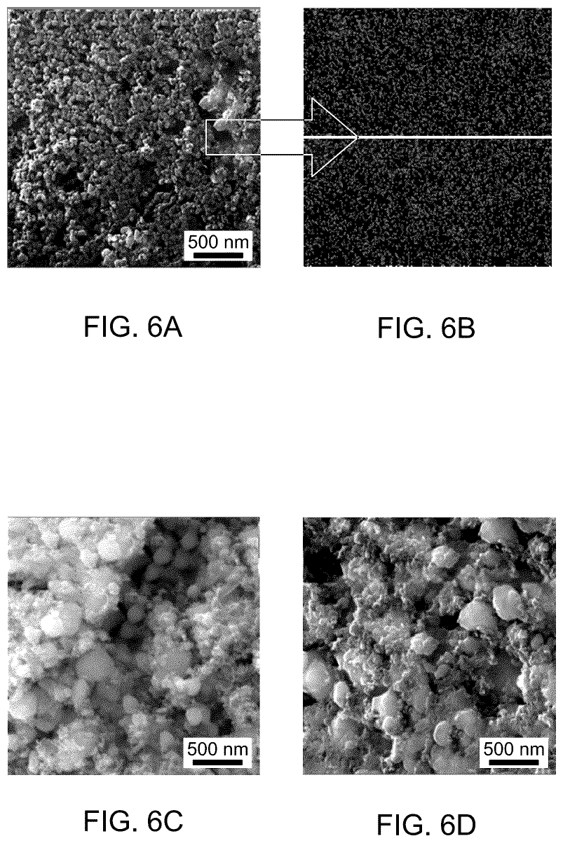

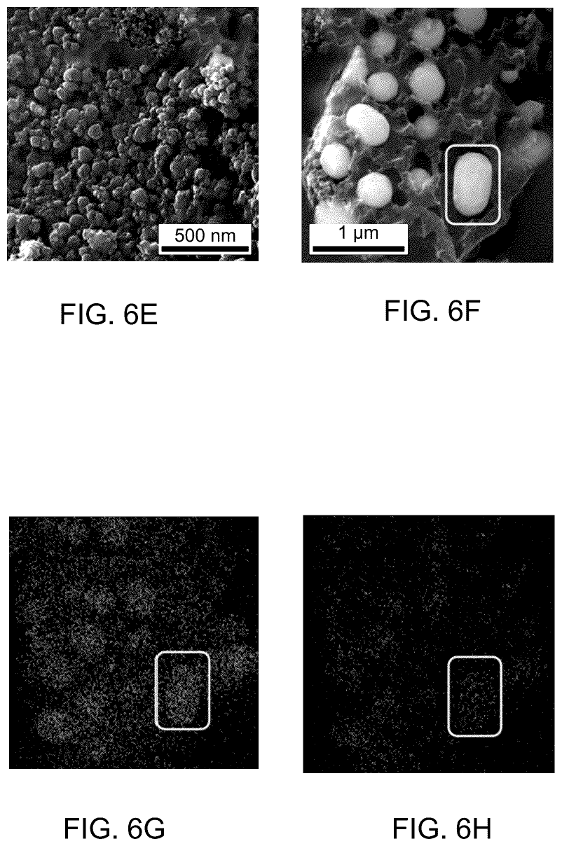

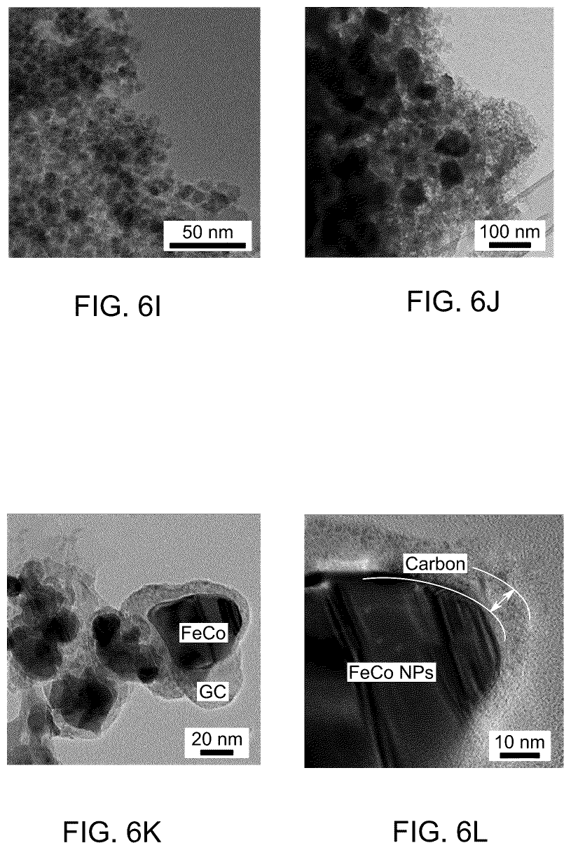

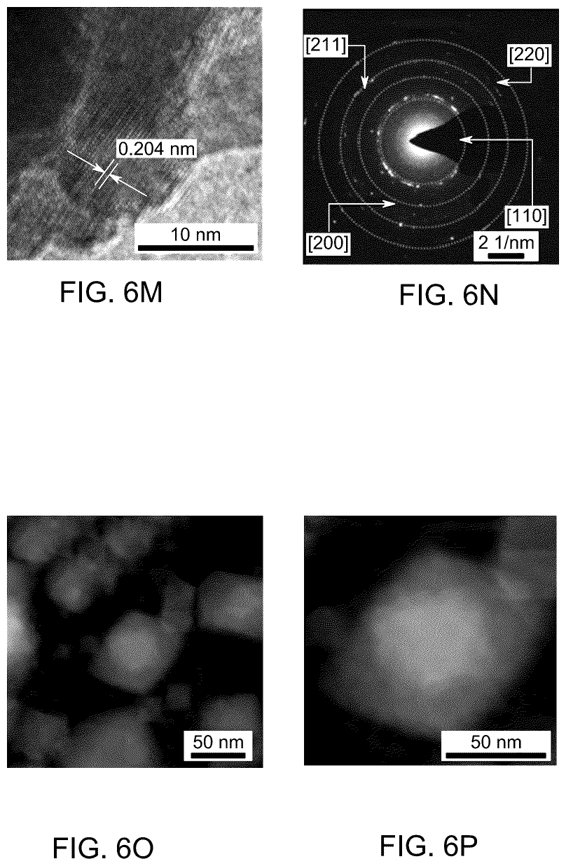

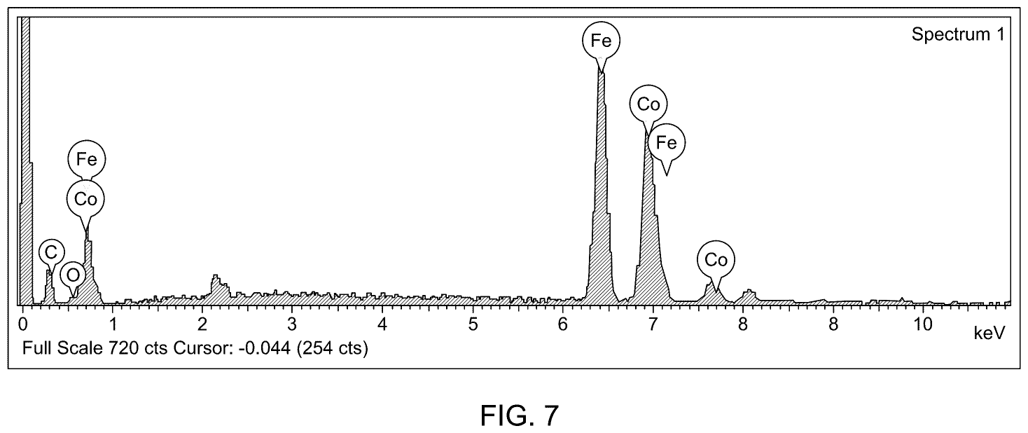

The FESEM images of FeCo@GC (1:1) at 600° C. are depicted in FIG. 6A, and the FESEM-EDS images of Fe and Co, are depicted in FIG. 6B. The Fe—Co formation mechanism at 600° C. with 3 hours of heating time was investigated using Co as the limiting reactant. The FESEM images of FeCo@GC (2:1), and FeCo@GC (3:1) at 600° C., are depicted in FIG. 6C and FIG. 6D, respectively. The SEM images reveal an increase in the Fe—Co particle size to 50 nm with increased inhomogeneity on increasing the temperature to 800° C. in FIG. 6E. With further increase of the temperature to 900° C., the smaller Fe—Co nanoparticles disappear, resulting in the 200 nm particles forming a birds' nest-like structure in FIG. 6F. The composition of the resulting catalyst was confirmed by FESEM-EDS (FIG. 7). FIG. 6G shows FESEM-EDS of Fe of FeCo@GC (1:1) at 900° C. and FIG. 6H shows FESEM-EDS of Co of FeCo@GC (1:1) at 900° C. The loss of smaller Fe—CO particles is attributed to Ostwald Ripening (OR), which is driven by the free energy difference and local adatom concentration. Ostwald Ripening led to a loss of active surface area of Fe—Co because the active surface area of the particles decrease as the particle size increases.

The TEM images in FIGS. 6I-6L show the morphologies of the as-prepared catalysts. The TEM image in FIG. 6I shows that pure Fe₃O₄ is in the form of finely distributed spherical particles (3-5 nm). When the alloy was formed, the particle sizes of FeCo@GC became larger, reaching 37-40 nm (FIG. 6J), which further increased to 200 nm as the temperature was increased to 900° C., as shown in SEM in FIG. 6F. Besides, in FeCo@GC, the nano-alloy was evenly distributed over the entire area of the specimen. This conclusion was supported by the elemental mapping conducted using SEM (FIG. 6B). The Fe—Co alloy appears in the darker core areas, and several layers of the graphitic carbon appear as the lighter zone around the Fe—Co alloy in the TEM images (FIGS. 6K-6L). The inter-planar distances (d-spacing) of 0.204 obtained using the data on lattice fringes acquired by a high-resolution transmission electron microscopy (HRTEM) identify the facet (110) of Fe—Co (FIG. 6M).

The FeCo@GC composition was confirmed by scanning electron microscopy/energy dispersive spectroscopy (SEM-EDS) data (FIG. 7). The selected area electron diffraction (SAED) results obtained using Bragg reflection data also yield a lattice spacing, d-value, of 0.204 nm corresponding to the <110> plane of Fe—Co (FIG. 6N). The differences in scanning transmission electron microscopy with high-angle annular dark field (STEM-HAADF) images in FIGS. 6O-6P of the brightness between the middle (core material) and edge (shell material (graphitic carbon)) regions of the particles demonstrates the core-shell type structure of the FeCo@GC catalysts.

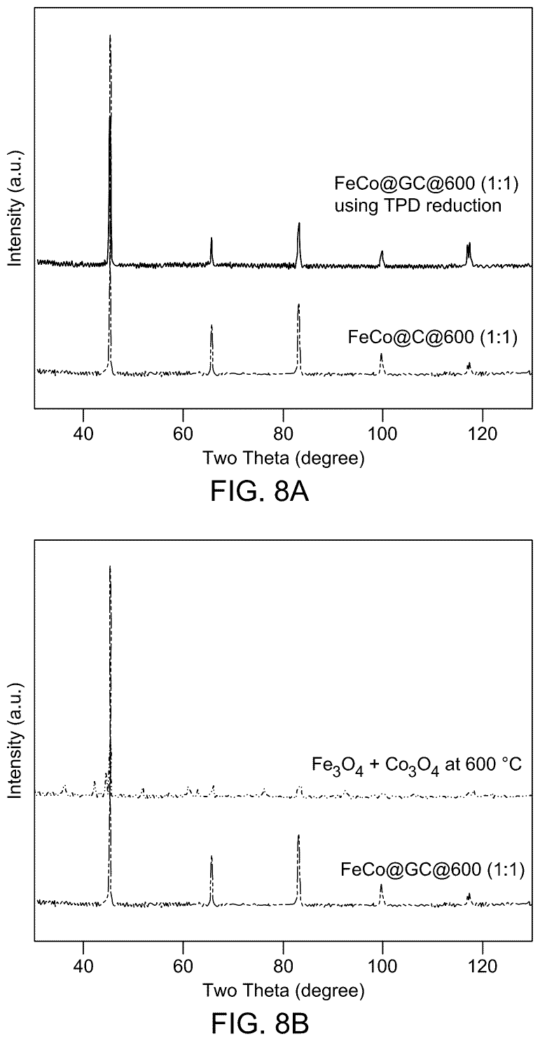

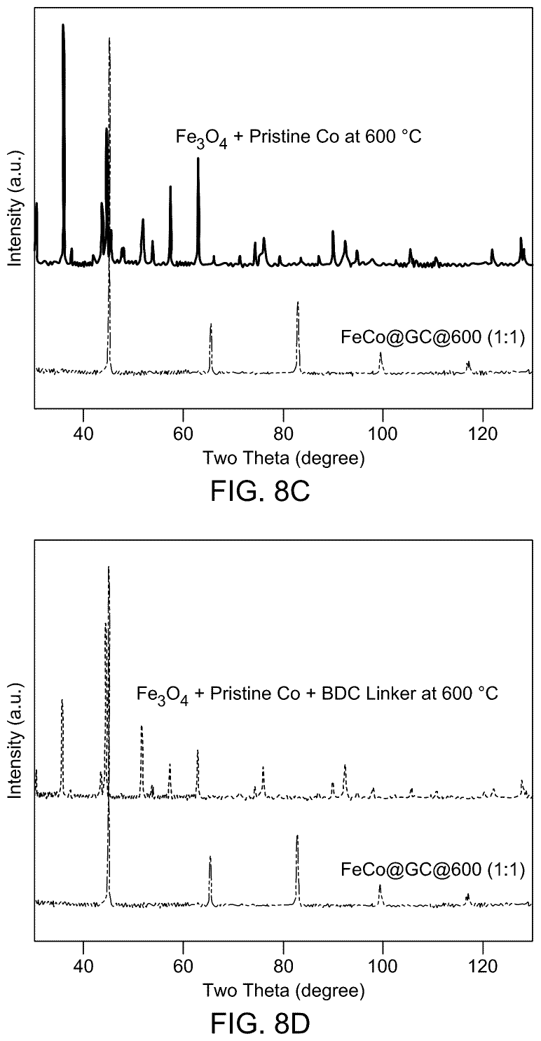

FIGS. 8A-8C show the reactions of equal atomic percentages of cobalt in cobalt oxide (Co₃O₄), pristine Co, and cobalt nitrate (Co(NO)₃) with Fe₃O₄ evaluated under the same reaction conditions. When Co₃O₄ was used as the source of Co, XRD showed that only a minimal amount of Fe₃O₄ and Co₃O₄ (approx. 20% each) reacted to form Fe—Co, and the rest remained unreacted shown in FIG. 8D. When pristine Co was used as the source of Co instead of Co₃O₄ and reacted with Fe₃O₄, almost 90% of the pyrolyzed product contained both unreacted Fe₃O₄ and metallic Co together with a negligible amount of Fe—Co as shown by XRD shown in FIG. 8C. Surprisingly, the addition of carbon sources in the form of 1,4-benzenedicarboxylic acid (BDC) (402), a linker used in MOF-71 (404), into the mixtures containing Fe₃O₄ (406), and pristine Co, no noticeable change in the formation of Fe—Co was observed and depicted in FIG. 8D. It is known that pyrolysis of nitrogen-containing zeolitic imidazolate framework (ZIF)-67 (Co) at 800° C. produces nitrogen-doped carbon-wrapped Co₃O₄ (20 nm) on Fe₃O₄ microcrystals (450 nm). [Millward, A. R.; Yaghi, O. M. Metal-Organic Frameworks with Exceptionally High Capacity for Storage of Carbon Dioxide at Room Temperature. J. Am. Chem. Soc. 2005, 127 (51), 17998-17999, incorporated herein by reference in its entirety]. Hence, the role of the MOF-71 (Co) framework in forming Fe—Co particles cannot be ruled out. However, it is also known that MOF-71 (Co) consists of infinite chains of corner-sharing octahedral metal ions (i.e., Co) linked to four parallel chains via BDC. In addition, MOF-71 (Co) was also known to have poor electrical conductivity and was redox silent. Considering that the standard reduction potential of E_{H, Fe(II)/Fe(0)}=−0.44 V was very close to that of cobalt (Co_{H, Co(II)/Co(0)}=−0.28), the galvanic effect might not play a role here. The reduction of Fe₃O₄ to the metallic Fe⁰ state to form Fe—Co might be triggered by reduced Co metal surrounded by carbon. This phenomenon could be enhanced by the homogeneous dispersion of the ultrasmall Fe₃O₄ particles (3-5 nm) in the porous and layered architecture of MOF-71 (Co). Also, the slow deconstruction of the carbon framework on the surface of the combined metal, Fe—Co, in the form of graphitic carbon under pyrolytic conditions might result in the formation of the alloy under this unique environment.

This scheme shows the importance of the Co-MOF precursor to forming the magnetically recoverable catalyst. Because the standard reduction potentials of Fe and Co are similar, formation of a FeCo alloy is unlikely to occur in the absence of the carbon framework of the MOF.

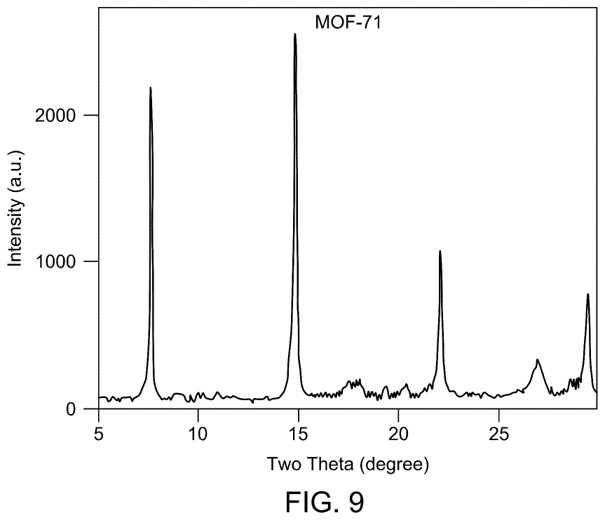

FIG. 9 shows the crystalline nature of MOF-71 was evident from its diffraction pattern. Furthermore, increasing MOF-71 (Co) content shifted the peaks slightly towards a higher angle than the XRD pattern of pure Fe—Co. This might be due to the lattice strain caused by substituting a higher number of relatively larger Fe atoms (atomic radius r_{Fe}=0.172 nm) with the smaller Co atoms (r_{Co}=0.125 nm), expanding the Fe—Co unit cell. Furthermore, pure Fe—Co was formed only when the atomic percentage of Fe:Co was equal, whereas lowering the cobalt content from 1:1 to 3:1 leads to the oxidation of Fe—Co to form CoFe₂O₄. The Fe—Co unit cell undergoes oxidation in the abundant oxygen of Fe₃O₄ and the linker of the MOFs to form cobalt ferrite, CoFe₂O₄, which was detected by FTIR with the appearance of Fe—O vibrational frequency at 582 cm⁻¹.

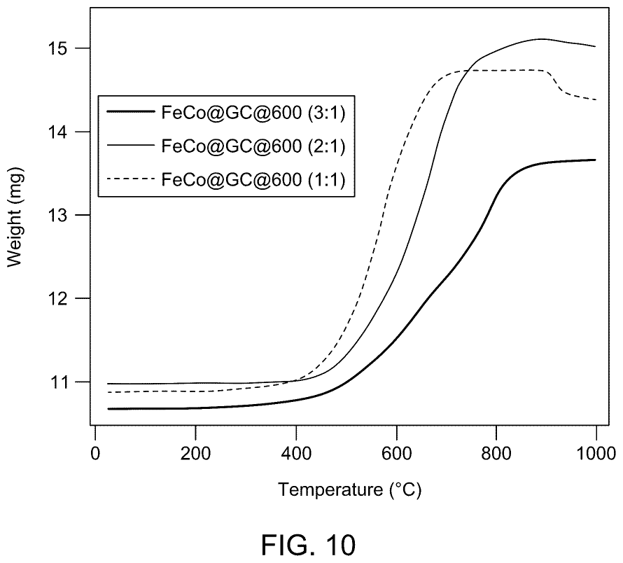

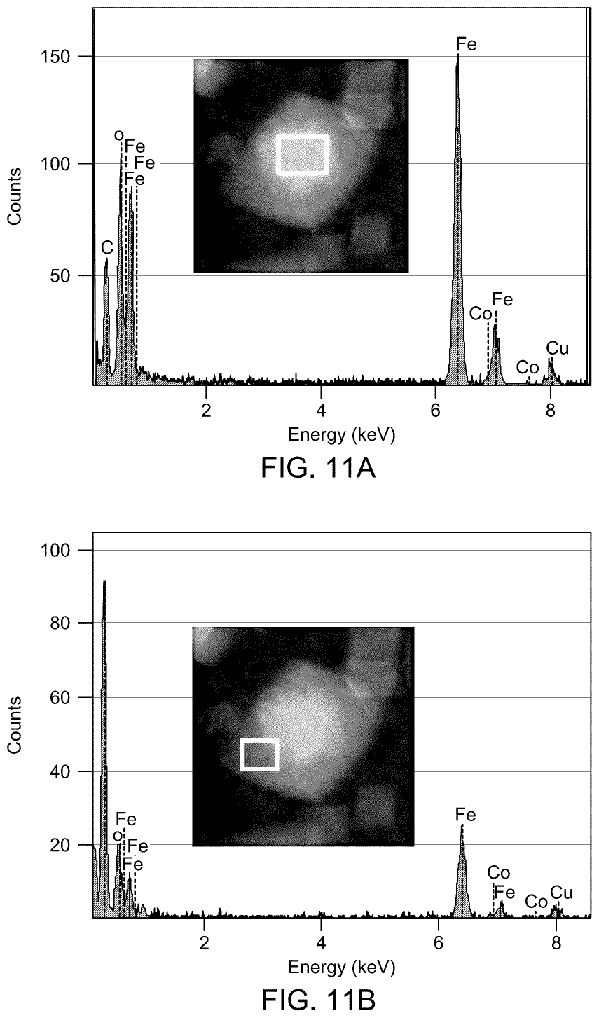

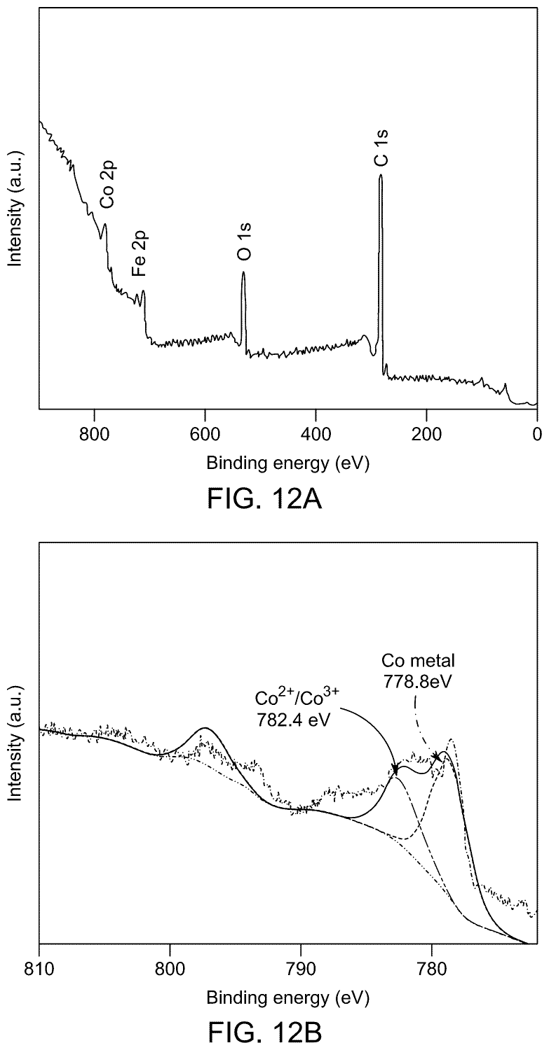

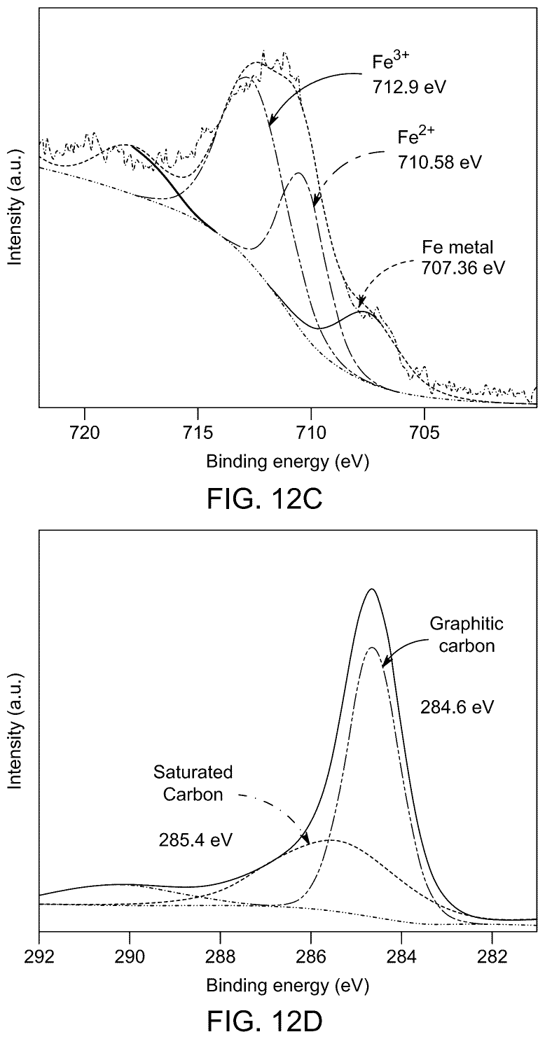

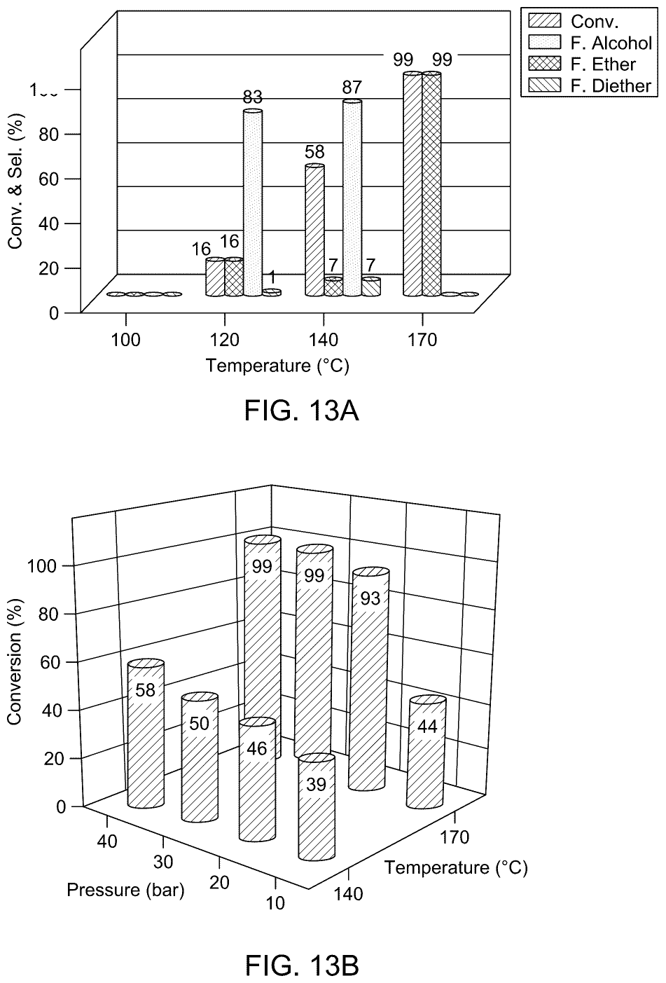

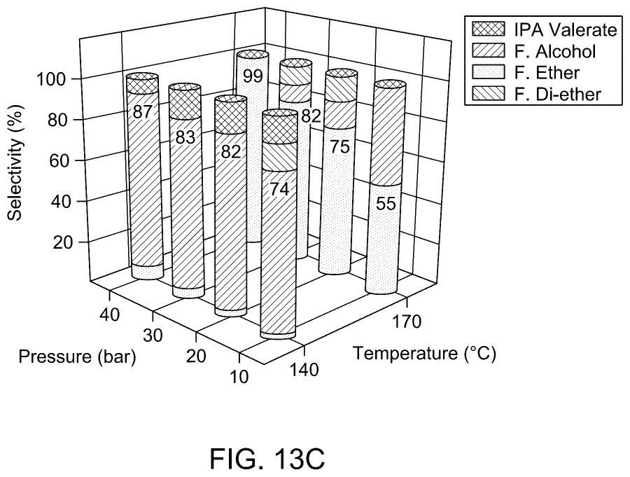

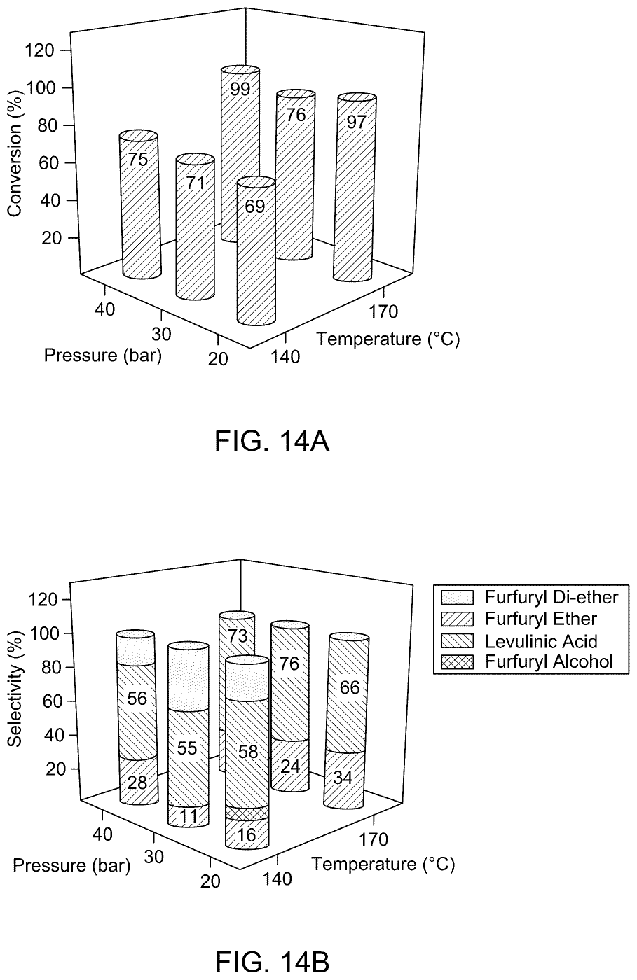

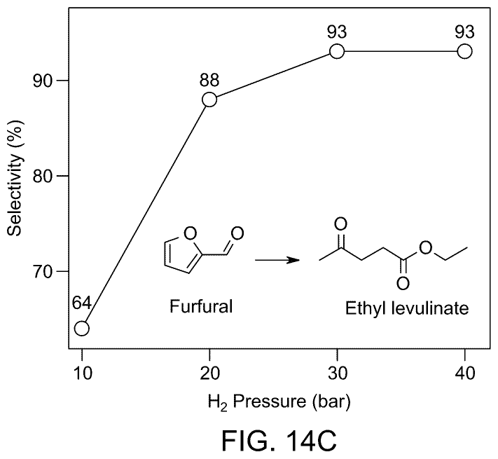

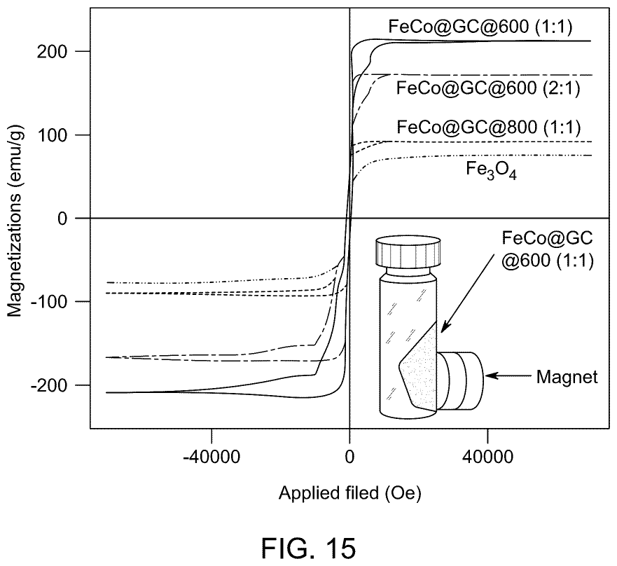

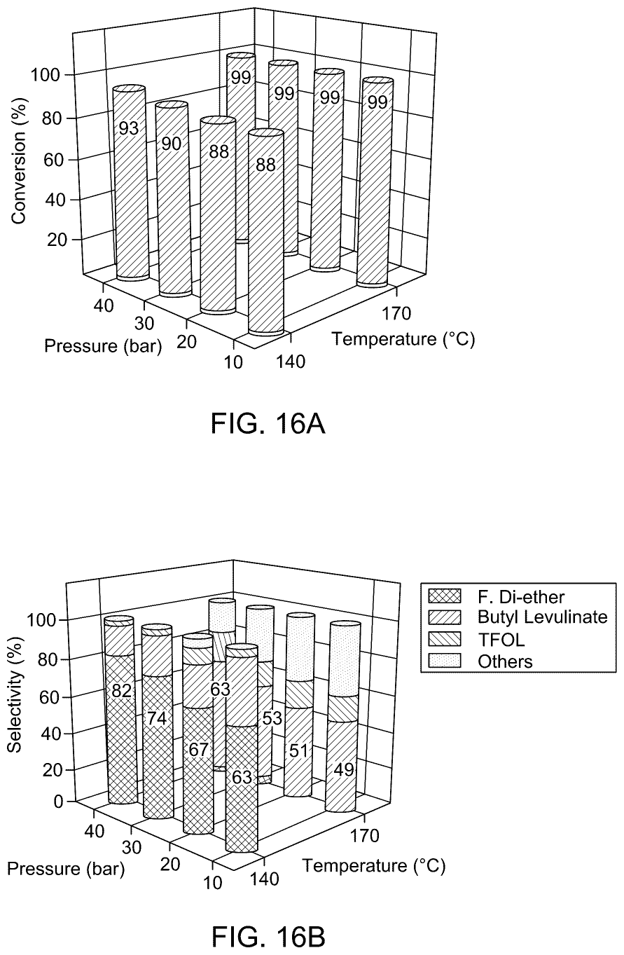

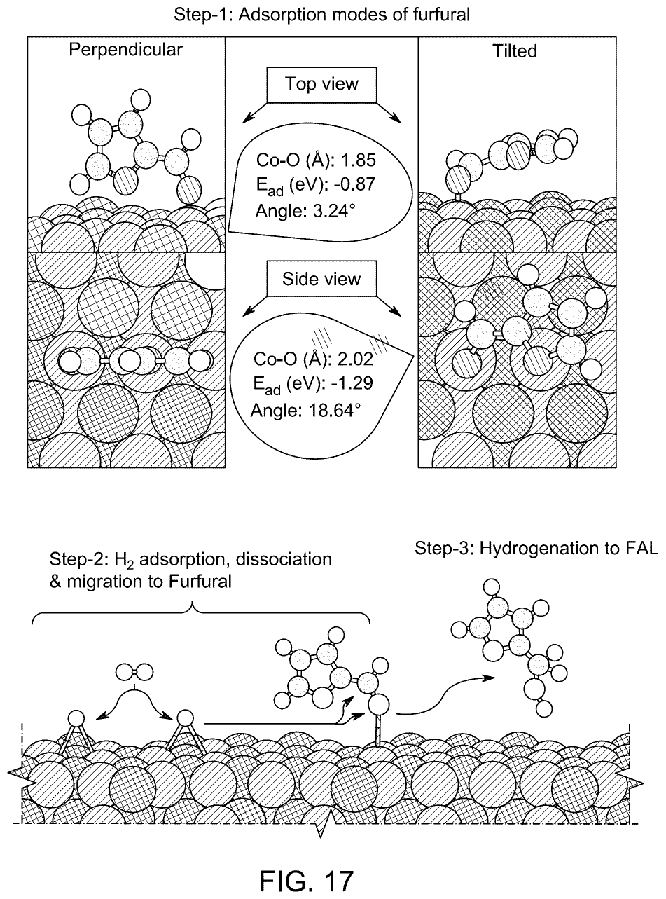

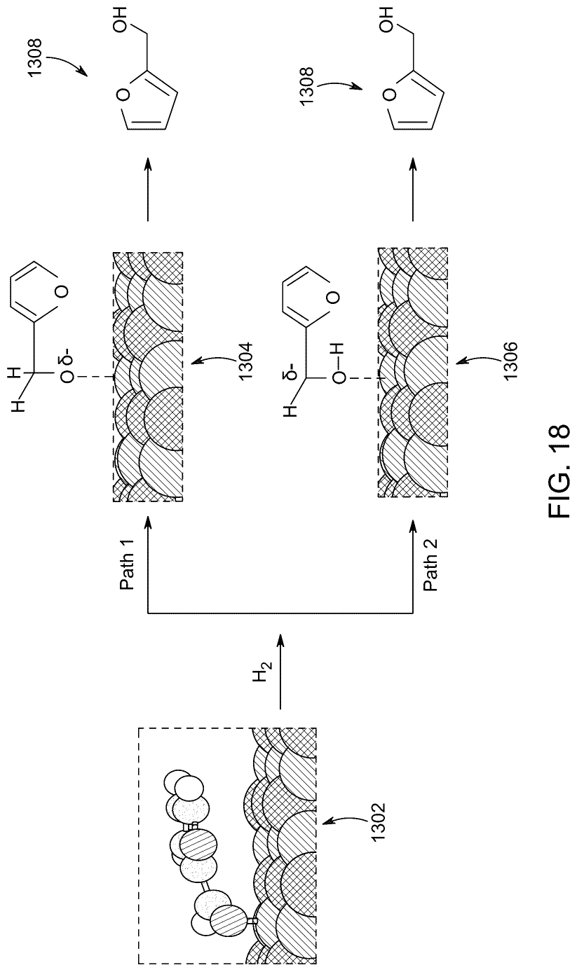

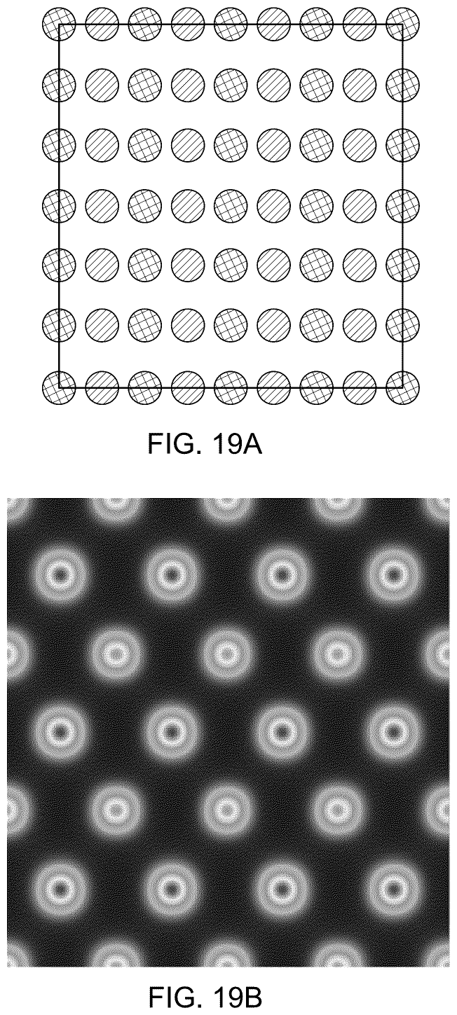

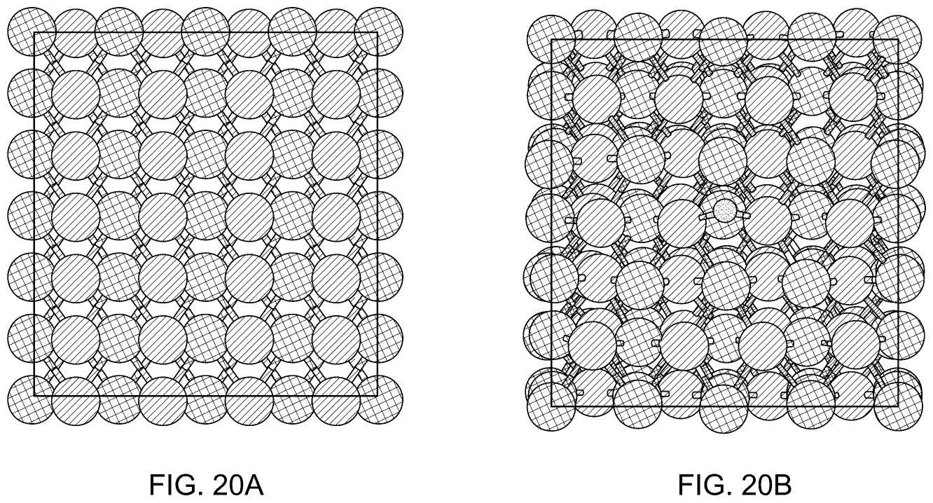

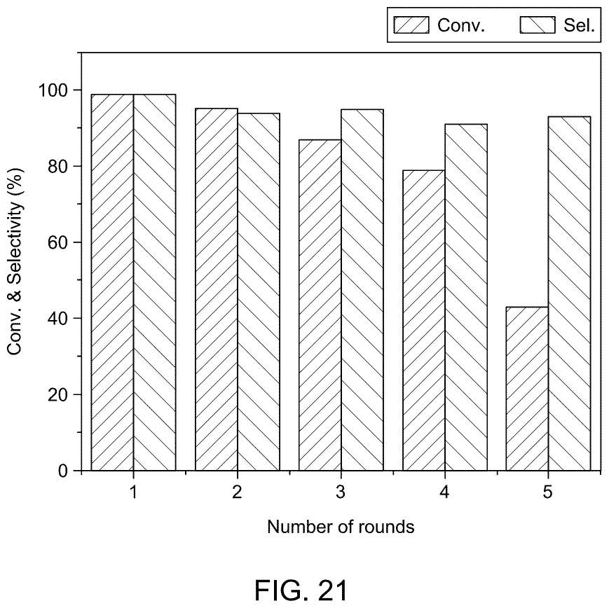

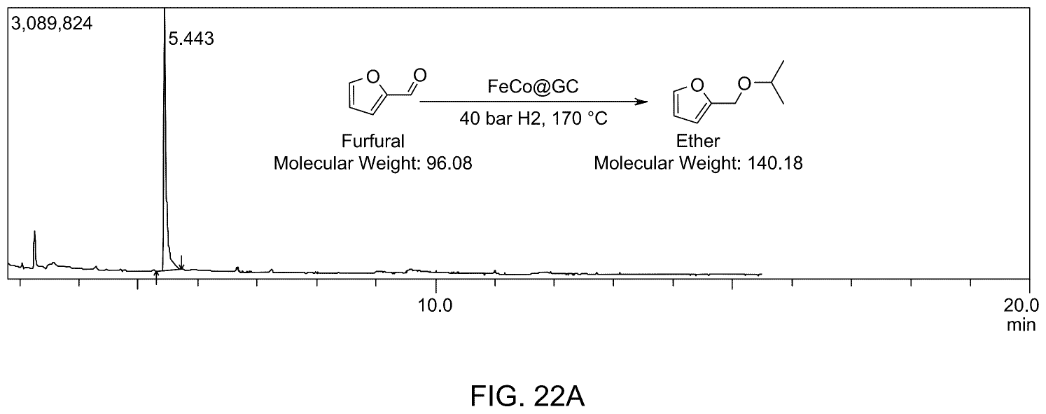

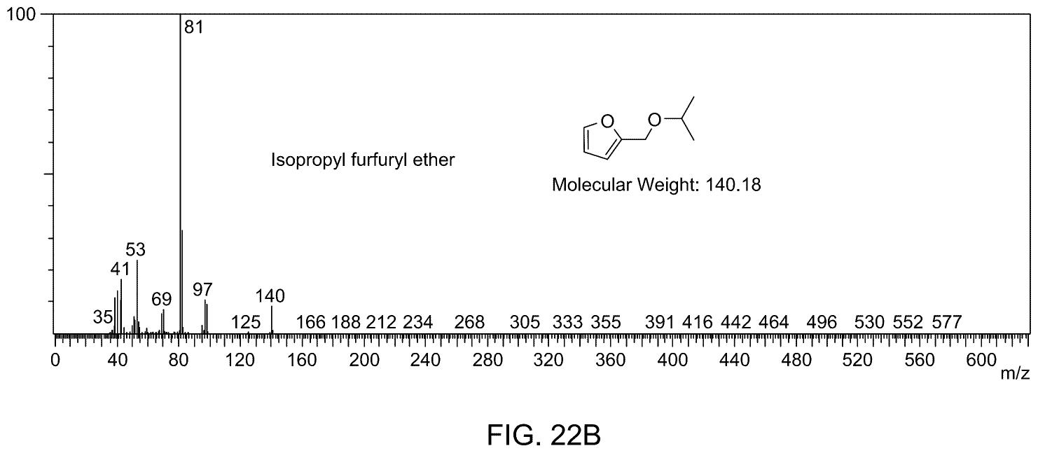

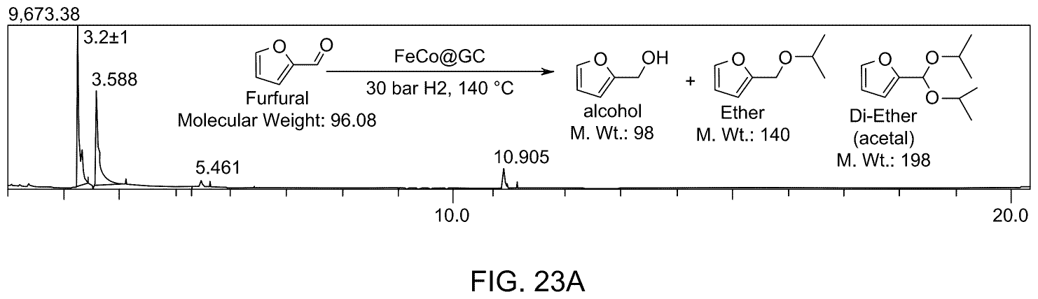

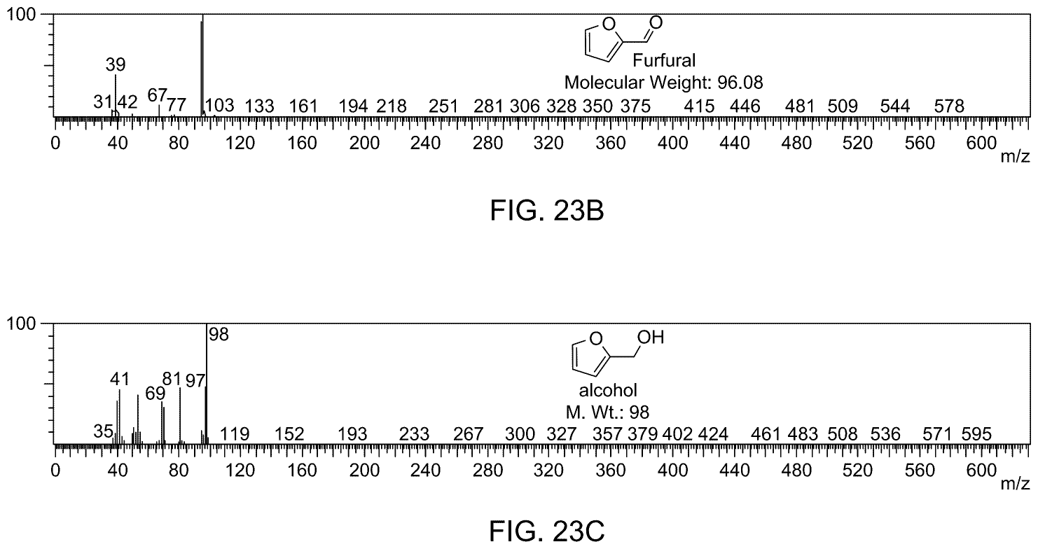

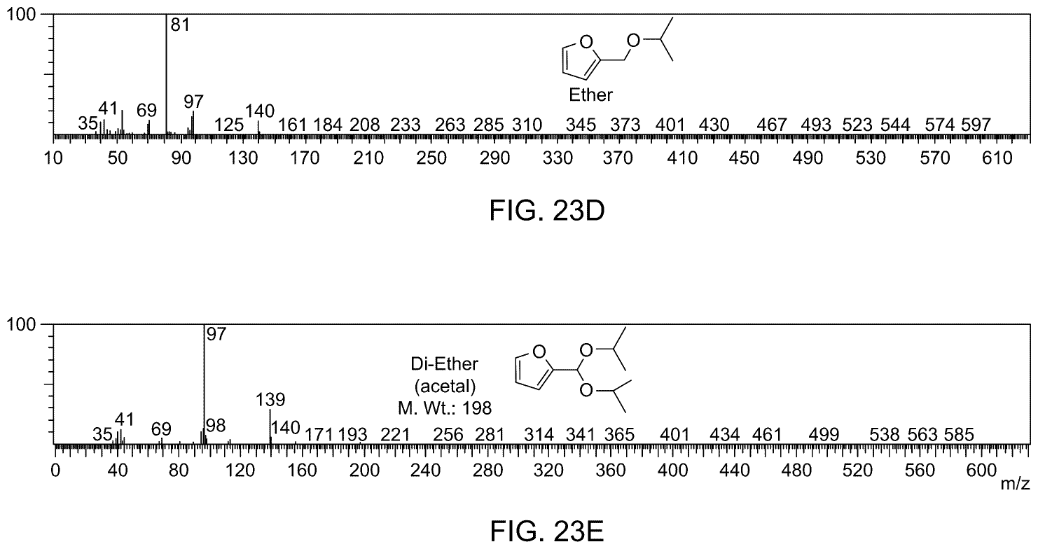

On increasing the pyrolysis temperature to 900° C., only Fe—Co signatures were detected with peaks broadening, indicating the smaller crystallite size, when the atomic percentage of Fe and Co was equal. A diffraction peak of carbon was not detected in XRD, most likely due to its amorphous nature. Furthermore, the TGA in air showed an apparent weight gain due to the oxidation of their respective metal oxides. This was further supported by TGA residue analysis, shown in FIG. 10, by XRD, which reveals the formation of a mixture of Fe₃O₄ and Co₃O₄. Generally, a larger excess of Fe results in a smaller weight gain and a higher temperature on-set for oxidation.

FIG. 11A and FIG. 11B show the co-existence of Fe and Co in the core. The higher concentration of carbon at the edges is confirmed by scanning electron microscopy with energy dispersive x-ray spectroscopy (STEM-EDX).

X-ray photoelectron spectroscopy (XPS) was used to obtain further information on the electronic structure and surface composition of FeCo@GC, and the XPS results are shown in FIGS. 12A-12B. The global survey confirms the presence of Fe, Co, and C (FIG. 12A). The oxidation states of surface species and the interaction between Fe and Co were investigated. The peaks at 778.8 electron volts (eV) and 782.4 eV (FIG. 12B) in pristine Co 2p are attributed to Co⁰ 2p_{3/2} and Co⁺²/Co³⁺ 2p_{3/2}, respectively. In FeCo@GC, the peak due to Co⁰ 2p_{3/2} shifts to a lower binding energy of 0.6 eV, indicating the enhanced electronegativity of Co in FeCo@GC, which was further confirmed by the DFT calculations. The peak at binding energy 707.4 eV was attributed to metallic Fe 2p in the Fe—Co core, formed during the pyrolysis of MOF-71 and Fe₃O₄ (FIG. 12C). Peaks at 710.6 and 712.9 eV suggested the existence of Fe²⁺ and Fe³⁺ species, respectively. However, it was worth noting that in addition to the metallic Fe/Co species, a small amount of ionic species, such as Co⁺²/Co³⁺ from Co₃O₄, which contained two distinct types of Co ions, and Fe²⁺/Fe³⁺ from Fe₃O₄ of both metals are detected. This might be due to the thin layer of high-valent oxides magnetically coupled with Fe—Co nanoparticles making the zero valent Fe/Co less detectable in XPS. In FIG. 12D, the peak at 284.6 was assigned to C 1s of the graphitic carbon.

FIG. 15 shows room temperature hysteresis loops of FeCo@GC nanoparticles with a different composition at different pyrolysis temperatures. The hysteresis loops of FeCo@GC alloy measured at 300 kelvin (K) indicated that saturation magnetization (Mₛ), the value when the haphazardly arranged molecular magnets within the core material become aligned with each other due to the applied magnetic field, is 213.70 electromagnetic units per gram (emu g⁻¹) for a specimen with 1:1 ratio of Fe:Co, which was higher than that of pure magnetite (77.56 emu g⁻¹). This is attributed to the combination of 3d metals (Fe and Co) leading to strong spin-orbit coupling, resulting in enhanced saturation magnetization. The data also showed that the magnetization pattern depended on the metal content. The magnetic susceptibility was higher at the 1:1 ratio of Fe and Co, possibly due to the homogeneous atomic distribution of iron and cobalt within the individual nanoparticles, compared to when the Fe:Co ratio is 2:1 (171.6 emu g⁻¹). Interestingly, the saturation magnetization (Ms) of FeCo@GC pyrolyzed at a higher temperature was disadvantageously lower (91.25 emu/g) than that of the alloy prepared at a lower temperature (600° C.).

### Catalysis

FIG. 13 shows the effects of reaction conditions on furfural hydrogenation. Furfural hydrogenation was performed using a branched alcohol, such as isopropyl alcohol (IPA), to determine the appropriate reaction conditions (FIG. 13A). Etherification using IPA produces the best results at higher temperatures. In comparison, at lower temperatures, the formation of furfuryl alcohol (FAL) is favored. FIGS. 13B-13C shows the effect of H₂ pressure on the conversion and selectivity. At 140° C. and 10 bar hydrogen pressure, the conversion (FIG. 13B) of furfural is 39%, while the selectivity for FAL is 74% (FIG. 13C). The highly polar solvent, isopropanol, might decrease the rate of adsorption of moderately polar furfural on the surface of FeCo@GC at lower pressure. When the temperature was increased from 140 to 170° C. at the same pressure of 10 bar, furfural conversion increased to 44% (FIG. 13A). This might be due to the mobility of the furfural molecule increasing due to the lowering of viscosity at the higher temperature and thereby enhancing the chances of adsorption of the furfural molecule on the surface of the bimetallic catalyst. At 40 bars, the conversion improved (58%) with 97% of FAL production (FIG. 13A). This could be due to the higher dissolution of hydrogen at higher pressure at 140° C. Interestingly, selectivity was markedly different at an elevated temperature (FIG. 13B). For instance, at 10 bars, formations of isopropoxy furfuryl ether (55%) predominated over the formation of FAL (34%).

The increasing selectivity trend with increasing pressure continued, and finally, >99% conversion with >99% isopropoxy furfuryl ether selectivity was achieved at 170° C. under 40 bars of H₂. The synergistic effects of the two metals in the FeCo@GC construct at a higher hydrogen pressure of 40 bar may lead to the conversion and the creation of new reaction pathways to form ether instead of FAL.

However, this reaction proceeded at a much lower rate, taking 20 hours to complete the reaction. Published results indicated that polar solvents enhanced the adsorption of relatively non-polar reactants and vice-versa. [Merlo, A. B.; Vetere, V.; Ruggera, J. F.; Casella, M. L. Bimetallic PtSn Catalyst for the Selective Hydrogenation of Furfural to Furfuryl Alcohol in Liquid-Phase. Catal. Commun. 2009, 10 (13), 1665-1669, incorporated here by reference in its entirety] Therefore, the selective reactivity towards the reduction of the carbonyl functional group instead of the furanic ring might be due to the adsorption of the furfural on the FeCo@GC nano-alloy through the lone pair of oxygen of the carbonyl group pushing the furanic ring away from the nano-alloy due to the effective repulsion between π-electron cloud and the metal. This hypothesis was further supported by the density functional theory (DFT) studies of the preferential adsorption of furfural on the surface of the FeCo@GC alloy.

A linear alcohol such as ethanol was used to investigate the wider applicability of the method (FIGS. 14A-14C). At 60° C. under 40 bar hydrogen, FeCo@GC (1:1) catalyst produces >96% diethoxy furfuryl ether (acetal) selectivity with low furfural conversion (28%) in 20 hours. The hydrogenation at 100° C. shows 40% conversion with 99% diethoxy furfural (DOF) formation. A significant amount of levulinic acid (LA) (73%) was produced at a higher temperature in 2 hours. LA was formed due to the decomposition of FAL, which was also detected in the hydrogenation reaction. Furthermore, the effect of hydrogen pressure was also studied at two different temperatures in (FIGS. 13A-13C). At 140° C., under 20 bars of hydrogen, the reaction produces LA with a 58% selectivity and a conversion of 69% in 2 hours. A noticeable change in the selectivity was not observed at 140° C. with a further increase of the H₂ pressure. However, at 170° C., a quantitative conversion was achieved at 20 bars of hydrogen with the formation of 66% LA. With further increased hydrogen pressure to 30 bars, selectivity for LA reached a maximum of 73% with a 99% conversion. This was probably due to higher hydrogen solubility producing a higher amount of FA and the subsequent hydrolysis of FA, forming LA. Surprisingly, when anhydrous ethanol was used, the catalyst was more active toward forming levulinate instead of LA. For instance, conversion improved from 69 to 99% with 88% ethyl levulinate selectivity when the temperature is 140° C. at 20 bar H₂ (FIG. 14A). A slight improvement (93%) in the selectivity for ethyl levulinate (93%) was observed at 30 bars and stays constant at 40 bars (FIG. 14B).

To investigate the hydrogenation of the furfural in the presence of n-butanol, a noticeable effect of H₂ pressure ranging from 10-40 bars was not observed on the conversion of the furfural, which reached 93% at 40 bars at 140° C. Also, the selectivity towards dibutoxy furfuryl ether was increased from 63% to 82% as the hydrogen pressure was increased from 10 to 40 bars. The furfural was fully converted at 170° C. under different hydrogen pressure with a butyl levulinate selectivity. For instance, selectivity for butyl levulinate reached 49%, and the trend continued with increasing the H₂ pressure, with selectivity reaching 63% in 20 hours. Notably, 8-10% of the over-hydrogenated product, tetrahydrofurfural, was detected in all reactions starting from 10 bars of hydrogen. These results indicated that at higher temperatures, long-chain alcohol might be converted to its corresponding levulinate.

FIG. 15 shows room temperature hysteresis loops of FeCo@GC nanoparticles with a different composition at different pyrolysis temperatures. The hysteresis loops of FeCo@GC alloy measured at 300 kelvin (K) indicated that saturation magnetization (Ms), the value when the haphazardly arranged molecular magnets within the core material become aligned with each other due to the applied magnetic field, is 213.70 electromagnetic units per gram (emu g⁻¹) for a specimen with 1:1 ratio of Fe:Co, which was higher than that of pure magnetite (77.56 emu g⁻¹). This is attributed to the combination of 3d metals (Fe and Co) leading to strong spin-orbit coupling, resulting in enhanced saturation magnetization. The data also showed that the magnetization pattern depended on the metal content. The magnetic susceptibility was higher at the 1:1 ratio of Fe and Co, possibly due to the homogeneous atomic distribution of iron and cobalt within the individual nanoparticles, compared to when the Fe:Co ratio is 2:1 (171.6 emu g⁻¹). Interestingly, the saturation magnetization (Mₛ) of FeCo@GC pyrolyzed at a higher temperature was disadvantageously lower (91.25 emu/g) than that of the alloy prepared at a lower temperature (600° C.).

FIGS. 16A and 16B show the overall conversion efficiencies and product selectivity at 140° C. and 170° C. as the hydrogen gas pressure in the reactor changes, respectively. At 140° C. the overall conversion efficiency averaged 90% and approached an average of 99% at 170° C. as FIG. 16A shows, regardless of hydrogen pressure. However, the selectivity increased as hydrogen pressure increased at both temperatures according to FIG. 16B. For example, at 140° C. the selectivity for furfuryl di-ether increased from 63% to 82% as the pressure increased from 10 bar to 40 bar. A similar pattern emerged at 170° C. for butyl levulinate.

### DFT Studies

A simulation was conducted to clarify the nature of furfural adsorption on Fe—Co (110) surfaces. Due to the differences in electronegativity between Fe (1.8) and Co (1.9) on the Pauling electronegativity scale, the formation of bimetallic Fe—Co (110) structures caused a redistribution of charges between Fe and Co in Fe—Co. Bader charge transfer indicates that 0.21e was transferred from Fe to Co.

FIG. 17A-17B shows that the hydrogen atom acts as a bridge between two Co atoms as it is attracted to both. The attraction between two Co atoms consistently increased as the electronegativity of the adsorbent and the electrostatic nature of all hydrogen bonds were well known. Due to the higher electronegativity of cobalt in Fe—Co, hydrogen bridges were formed between two cobalt atoms. Consequently, hydrogen dissociation was more likely to occur on Co sites of Fe—Co (110).

FIG. 17 shows that the total electron density of Co in Fe—Co was marginally higher than that of Fe. The charge redistribution in Fe—Co (110) improved the catalytic properties of Co relative to those of Fe. Hydrogen was initially adsorbed on top of Fe in Fe—Co to examine the interaction between H and Fe—Co (110). However, the hydrogen atom bonded to Fe in the initial structure, before geometry optimization, was transferred to Co during the geometry optimization of FeCo (110). This hydrogen migration from iron to cobalt indicated a strong interaction between hydrogen and cobalt, with an adsorption energy of −2.65 eV. Two different adsorption orientations of the furfural on Fe—Co (110) surfaces were designed, as shown in FIG. 17, to investigate the interaction between furfural and Fe—Co (110). Based on in-situ FT-IT experiments, the adsorption energy of tilted and perpendicular adsorption orientations on Fe or Co sites of Fe—Co (110) were primarily considered. For both orientations, the interaction of the furfural with Fe and Co atoms in Fe—Co (110), which have distinct charge distributions, was estimated separately. The catalytic selectivity of the furfural relative to other products was primarily determined by the adsorption orientation of C═O on the catalyst surface, which was strongly correlated with the adsorption energy. The adsorption energy of the furfural on Fe—Co (110) surfaces was estimated using the following equation:

$${E}_{ads} = {E}_{Furfural @ FeCo ( 110 )} + {E}_{FeCo ( 110 )} + {E}_{Furfural}$$

DFT calculations demonstrated that the O atom of the furfural forms a covalent bond with either Fe or Co in both orientations of the molecule on the Fe—Co (110) surface. Adsorption energies for the perpendicular orientation of the furfural on the Fe and Co sites of Fe—Co (110) were −1.63 eV and −0.87 eV, respectively. In contrast, the adsorption energies for the tilted orientation of furfural on Fe and Co sites were −3.58 eV and −1.29 eV, respectively (Table 1). The calculated adsorption energies for the perpendicular and tilted orientations of the furfural on Fe (110) are −0.33 eV and −2.71 eV, respectively (Table 1). According to the adsorption energies, the presence of Co in Fe—Co structures increased the catalytic activity of Fe—Co (110) structures by providing a clear furfural adsorption pathway for its activation and hydrogenation. The calculated d-band center for Fe (110) and Fe—Co (110) surfaces indicated that the d-band center of Fe—Co (110) (−1.47 eV) was farther away than that of pure Fe (110) (−0.84 eV), indicating a downshift of the d-band center. According to published results, a metal with the d-band center energies between antibonding and bonding orbitals might be a good candidate for developing adsorbate-metal interactions. Alloying provided a unique method for tuning the d-band structure of catalytic sites, consequently influencing the surface's molecular adsorption. Both tilted and perpendicular orientations might reduce the interaction between the furan ring and the metallic surface, preventing the furan ring from getting activated and thereby promoting the hydrogenation of furfural on C═O on Fe—Co (110).

TABLE 1
shows data obtained from DFT calculations.

| | Adsorption Energy (eV) | | Pauling | | Charge per | |
|---|---|---|---|---|---|---|
| Sample | Tilted | Perpendicular | Electronegativity | | Atom (e) | |
| Fe (110) | −2.71 | −0.33 | Fe: | 1.8 | Fe: | 8.00 |
| FeCo (110)^{a} | −3.58 | −1.63 | Fe: | 1.8 | Fe: | 7.79 |
| FeCo (110)^{b} | −1.29 | −0.87 | Co: | 1.9 | Co: | 9.21 |

^{a}Furfural adsorbed on Fe site of FeCo (110) surface;
^{b}Furfural adsorbed on Co site of FeCo (110) surface.

### Mechanism

The furfural hydrogenation over a range of solid-supported catalysts was studied extensively. DFT calculations proposed a three-step mechanism for the catalytic hydrogenation of the furfural on FeCo@GC, as shown in FIG. 17: (i) adsorption of the furfural, (ii) adsorption and dissociation of H₂ followed by migration of the dissociated H atoms, and (iii) completion of the furfural hydrogenation on FeCo@GC. First, the furfural adsorbs on Co sites of Fe—Co surfaces through covalent bonds between Co and O of length 1.85 and 2.02 Å in perpendicular and tilted orientations, respectively. However, the angle (18.64°) and adsorption energy (−1.29 eV) obtained from the DFT calculations indicated that the interaction of the tilted orientation was more favorable than the interaction of the perpendicular orientation. The computed adsorption energies for Co and Fe sites indicated that Co has a higher reaction probability than Fe (Table 1). Moreover, furfural has two possible sites for hydrogenation, i.e., the five-membered furan ring and the carbonyl functional group. Hence, adsorption of the furfural on the catalyst preferentially through either of the two competitive reaction sites determined the hydrogenation product.

FIG. 18 shows that furfural (1808) tends to adsorb through the aldehyde functional group in different binding modes, including η¹ (O)-aldehyde or η² (C,O)-aldehyde (FIG. 18). Unlike noble metals (Pt or Pd), unsaturated compounds with a C—O functional group tend to adsorb on the surface of the Fe—Co catalyst via η¹ (O)-aldehyde through the lone pair of electrons of oxygen, consequently favoring the hydrogenation of C═O functional group instead of the furan ring. Further hydrogenation of η¹ (O)-aldehyde might produce either alkoxide (FIG. 18 Path 1) or hydroxyalkyl (FIG. 18 Path 2) intermediate depending upon the point of attack of H. DFT calculations indicated that the activation energy barrier for the hydroxyalkyl intermediate (1806) was lower than that for the alkoxide intermediate (1804), which promoted the reaction of the H atom with the O atom rather than the C atom of the carbonyl group. However, when the mode of adsorption was via η² (C, O)-aldehyde, as both C and O are involved in adsorption on the Fe—Co surface, further hydrogenation may produce the same hydroxyalkyl intermediate (1806) as η¹ (O)-aldehyde (Path 2) having FA. The two-atom binding mode involving η² (C,O)-aldehyde may be converted into η¹ (C)-aldehyde binding configuration at 140 or 170° C., which was thermodynamically favorable for the decarboxylation to produce the furan. However, furan was not detected in the entire course of the reaction. Hence, according to DFT calculations and experimental evidence, the furfural undergoes hydrogenation via η¹ (O)-aldehyde pathway (Path 2) to produce FA, and hydrogenation produces alkyl ether or alkyl levulinate. In Step 2, adsorption, and dissociation of H₂ on Co sites of the Fe—Co (110) surface, followed by migration of the dissociated H atoms, occur. Note that Co plays an active role in the adsorption and dissociation of H₂ molecule on the Fe—Co surface because of its high electronegativity (1.9) compared to that of Fe, due to which each Co accumulates an additional charge (0.21e) in FeCo (110). In Step 3, the hydrogenation of the furfural to the furfural alcohol occurred as the H atom migrating along the FeCo surface overcomes the chemical barrier to approach furfural, as schematically shown in FIG. 17. Because Co metal has a filled d-orbital, a repulsive force is present between its d-orbital and p-orbital of the furan ring of furfural.

FIG. 19A shows the charge redistribution between Fe and Co in the FeCo (110) plane. FIG. 19B shows the electron density contour map of the FeCo (110) plane. The charge resdistribution and electron density contour mapping indicates the redistribution and electron density is located around cobalt atoms and facilitates furfural hydrogenation.

FIG. 20A shows the adsorption of H on Fe before geometry optimization of the FeCo (110) crystal plane. FIG. 20B shows the formation of H bridges to Co atoms after geometry optimization of the FeCo (110) crystal plane. As described in the discussion of FIG. 17, hydrogen first adsorbs onto the Fe atoms of the FeCo (110) crystal plane and then later forms bridges to the more electronegative Co atoms.

### Reusability

In addition to their activity and selectivity, a highly advantageous property of heterogeneous catalysts is reusability, which enhances the prospect of practical applications. Moreover, the magnetic components introduced into the catalytic system strengthen the ability to separate it from the reaction system for reuse. Another requirement for the reuse of a catalyst is stability. Thus, the chemical stability FeCo@GC (1:1) catalyst was determined using isopropanol as the solvent under designed reaction conditions, and the results are shown in FIGS. 21A-21B. Fe—Co wrapped in the graphitic carbon retains good reactivity (79%) up to the 4th cycle (FIGS. 21A-21B) with unchanged selectivity at 170° C. under 40 bar H₂ and becomes unresponsive to the hydrogenation after the 6th cycle. This means that the porous carbon sheet was, undoubtedly, protecting the surface of the bimetal from poisoning due to the adsorption of the furfural or any of the other reaction products up to the 4th cycle. The surface and lattice structure of the reused catalyst were characterized using SEM and XRD. Significant damage to the surface or agglomeration was not detected. XRD shows that the diffraction pattern remains unchanged, indicating that the lattice remains intact and morphological changes do not occur under hydrogen pressure.

FIG. 22A shows gas chromatography (GC) and a mass spectrometry (MS) of the furfural hydrogenation using the FeCo@GC catalyst in isopropyl alcohol at 170° C. under 40 bar H₂. FIG. 22B shows MS spectra of the furfural hydrogenation using the FeCo@GC catalyst in isopropyl alcohol at 170° C. under 40 bar H₂. The absence of molecular fragments not attributable to molecular fragments in the desired product indicates that the reaction has a high overall conversion and efficiency rate.

FIG. 23A shows GC and MS spectra of the furfural hydrogenation using the FeCo@GC catalyst in isopropyl alcohol at 140° C. under 30 bar H₂. FIG. 23B shows GC and MS spectra of furfural during the furfural hydrogenation using the FeCo@GC catalyst in isopropyl alcohol at 140° C. under 30 bar H₂. FIG. 23C shows GC and MS spectra of furfural alcohol during the furfural hydrogenation using the FeCo@GC catalyst in isopropyl alcohol at 140° C. under 30 bar H₂. FIG. 23D shows GC and MS spectra of Isopropyl furfuryl ether during the furfural hydrogenation using the FeCo@GC catalyst in isopropyl alcohol at 140° C. under 30 bar H₂. FIG. 23E shows GC and MS spectra of diether (acetal) during the furfural hydrogenation using the FeCo@GC catalyst in isopropyl alcohol at 140° C. under 30 bar H₂, according to certain embodiments. Collectively, FIG. 22 A-B and FIG. 23A-FIG. 23E demonstrate that the hydrogenation process produces essentially only the intended product and very few impurities as evidenced by the absence of molecular fragments not attributable to groups in the desired product.

### EXAMPLES

The following examples demonstrate the method 50 for a process for producing a magnetically recoverable catalyst, as described herein. The examples are provided solely for illustration and are not to be construed as limitations of the present disclosure, as many variations thereof are possible without departing from the spirit and scope of the present disclosure.

### Catalyst Characterization Techniques

The X-ray diffraction (XRD) patterns were recorded on a Rigaku model Ultima-IV diffractometer employing Cu-Kα radiation (λ=1.5406 Å) at 40 kV and 25 mA over a 20 range between 2° and 130° (manufactured by Rigaku, Mar. 9, 2012 Matsubara-cho, Akishima-shi, Tokyo, 196-8666, Japan). All XRD patterns were recorded in an air atmosphere. The transmission electron microscopic (TEM) images were acquired at the Instituto de Nanociencia de Aragón (LMA-INA), University of Zaragoza, Spain, on a TEM (Titan, FEI) operated at 200 kV with a 4k×4k CCD camera (Ultra Scan 400SP, Gatan, 5794 W. Las Positas Blvd. Pleasanton, CA 94588. United States).

High-resolution transmission electron microscopic (HRTEM) images were obtained in an image-corrected Titan (FEI) at a working voltage of 300 kV. X-ray energy dispersive spectra (EDS) were obtained with an EDAX detector. The TEM samples were prepared by placing a drop of an ethanolic suspension on a copper grid and allowing it to dry at room temperature.

The Scanning Transmission Electron Microscopy-High Angle Annular Dark Field (STEM-HAADF) images were obtained using a Cs-probe-corrected Titan (ThermoFisher Scientific, formerly FEI, 68 Third Avenue. Waltham, MA USA 02451, United States) at a working voltage of 300 kV, coupled with an HAADF detector (Fischione, 9003 Corporate Circle Export PA, 15632. United States). The samples for SEM were prepared by applying ethanolic suspensions on single-sided alumina tape placed on alumina stubs. For the elemental analysis and mapping, energy-dispersive X-ray spectra (EDS) were collected on a Lyra 3 (Tescan, Grazhdanskiy Prospekt 11, Sankt-Peterburg, Rusko 195220, Czech Republic) attachment in the SEM.

The Magnetic Properties Measurement System-III-Superconducting Quantum Interference Device (MPMS-SQUID) was used to determine the magnetic susceptibilities at room temperature using a SQUID equipped with a 7-Tesla magnet with a temperature range of 1.8-400K.

The catalytic reactions were performed using Teflon-lined vessels in an autoclave from HiTech, USA (model M010SSG0010-E129A-00022-1D1101), (manufactured by Hitech solutions, 9350 SW 137TH Ave Unit 508 Sunrise, FL, 33351 United States) fitted with a pressure gauge and a mechanical stirrer.

The catalytic products were identified using a Shimadzu 2010 Plus (manufactured by Shimadzu Corporation, 1, Nishinokyo Kuwabara-cho, Nakagyo-ku, Kyoto 604-8511, Japan) gas chromatograph coupled with a mass spectrometer (GC-MS). The disappearance of the reactants and sequential appearance of the products were recorded in real-time, identifying the species in terms of their molecular ion (M+) by comparing and matching them with the Wiley library of the mass spectral database, in addition to the identification of mass fragmentation.

The Fe and Co concentration in the catalyst were determined using inductively coupled plasma optical emission spectrometry (ICP-OES; PlasmaQuant PO 9000-Analytik Jena, manufactured by Analytik Jena, Konrad-Zuse-Straße 1, 07745 Jena, Germany). The samples were first digested in a mixture of dilute HNO₃ and dilute HCl. Calibration curves were prepared for Fe and Co using standard solutions (ICP Element Standard solutions, Merck, 2000 Galloping Hill Rd, Kenilworth, NJ 07033, United States).

Surface chemistry was studied using an X-ray photoelectron spectroscope (XPS) equipped with an Al-Kα micro-focusing X-ray monochromator (ESCALAB 250Xi XPS Microprobe, Thermo Scientific, 168 Third Avenue. Waltham, MA 02451, United States). The chamber pressure was 2×10⁻⁹ torr. The binding energies are referenced to the C 1s line at 284.6 eV. Typical XPS survey spectra of the fabricated films and O1s, Co2p_{3/2}, and N1s core-level spectra for the nano-catalyst were obtained.

The Raman spectra were recorded using a Thermo Scientific DXR Raman spectroscope with a DXR 455 nm laser (manufactured by Thermo Scientific, 168 Third Avenue. Waltham, MA 02451, United States).

### Computational Methodology

Spin-polarized density functional theory (DFT), which is a generalized form of conventional DFT, as implemented in the Vienna Ab initio Simulation (VASP) code to study the catalytic properties of spin-polarized FeCo (110) structure was employed. Generalized gradient approximation (GGA) with the PBE function was chosen to illustrate the exchange correlation interaction, whereas the projected augmented wave (PAW) potentials were used to describe the core electrons. A kinetic energy cut-off of 400 eV on a plane wave basis was used throughout the calculations. Monkhorst Pack (MP) grid was used to sample the Brillouin zone (BZ) with a 2×2×1 k-mesh. Minimum energy was achieved using conjugate gradient optimization. DFT-D3 method was used to account for the van der Waals interactions in the DFT calculations. For structural relaxation, the convergence criterion of energy was taken as 1.0×10−5 eV, and the Hellmann-Feynman force on each atom was set to less than 0.01 eV/Å. Due to the periodic boundary condition, the distance between neighboring FeCo layers in each model was set at 15 Å along c-axis to avoid image-image interaction.

### Production Example 1: Synthesis of FeCo@GC Nano-Alloys

Magnetic nanoparticles like iron oxide (Fe₃O₄) in the 3-4 nm range were prepared according to the literature procedure reported elsewhere. Freshly prepared magnetite (350 milligrams (mg)) was ground with the mortar-pestle for 30 min. Then, a metal-organic framework (MOF)-71 (650 mg) was added, mixed, and ground for another 30 mins. The resulting mixture was pyrolyzed under a continuous flow of nitrogen (10 mL/min) gas, in a tubular furnace, with a ramp rate of 5 degrees centigrade per minute (° C./min); the temperature was held at 600° C. for 3 hours. The resulting catalyst is denoted as an iron-cobalt alloy encapsulated in a graphite carbon (FeCo@GC)@600 (1:1).

### Production Example 2

FeCo@C was prepared according to Example 1 except that Fe:Co atomic ratio was 2:1.

### Production Example 3

FeCo@C was prepared according to Example 1 except that the Fe:Co atomic ratio was 3:1.

### Production Example 4

FeCo@C was prepared according to Example 1 except that pyrolysis occurred at 800° C.

### Production Example 5

FeCo@C was prepared according to Example 1 except that pyrolysis occurred at 900° C.

### Example 1: Furfural hydrogenation

A furfural was hydrogenated using a teflon-lined autoclave fitted with a pressure gauge and overhead mechanical stirring. The furfural (0.5 mmol), anhydrous isopropanol (3 mL), and FeCo@GC catalyst were placed and flushed with H₂ three times and filled with 40 bars H₂. The reaction was performed by heating the autoclave to 170° C. with continuous stirring (500 revolutions per minute (rpm)). The reactor was cooled down and depressurized. The degree of conversion was determined by GC, and the products were identified by GC-MS.

### Example 2

Furfural hydrogenation was performed according to Example 1 except that the hydrogenation reaction temperature was 140° C.

### Example 3

Furfural hydrogenation was performed according to Example 1 except that the hydrogen pressure inside the reactor was 10 bar.

### Example 4

Furfural hydrogenation was performed according to Example 1 except that the hydrogen pressure inside the reactor was 20 bar.

### Example 5

Furfural hydrogenation was performed according to Example 1 except that the hydrogen pressure inside the reactor was 30 bar.

### Example 6

Furfural hydrogenation was performed according to Example 1 except that the alcohol was ethanol.

### Example 7

Furfural hydrogenation was performed according to Example 1 except that the alcohol was ethanol and the hydrogen pressure inside the reactor was 10 bar.

### Example 8

Furfural hydrogenation was performed according to Example 1 except that the alcohol was ethanol and the hydrogen pressure inside the reactor was 20 bar.

### Example 9

Furfural hydrogenation was performed according to Example 1 except that the alcohol was ethanol and the hydrogen pressure inside the reactor was 30 bar.

### Example 10

Furfural hydrogenation was performed according to Example 1 except that the alcohol was n-butanol.

### Example 11

Furfural hydrogenation was performed according to Example 1 except that the alcohol was n-butanol and the hydrogen pressure in the reactor was 10 bar.

### Example 12

Furfural hydrogenation was performed according to Example 1 except that the alcohol was n-butanol and the hydrogen pressure in the reactor was 20 bar.

### Example 13

Furfural hydrogenation was performed according to Example 1 except that the alcohol was n-butanol and the hydrogen pressure in the reactor was 30 bar.

### Example 14

Furfural hydrogenation was performed according to Example 2 except that the hydrogen pressure inside the reactor was 10 bar.

### Example 15

Furfural hydrogenation was performed according to Example 2 except that the hydrogen pressure inside the reactor was 20 bar.

### Example 16

Furfural hydrogenation was performed according to Example 2 except that the hydrogen pressure inside the reactor was 30 bar.

### Example 17

Furfural hydrogenation was performed according to Example 2 except that the alcohol was ethanol.

### Example 18

Furfural hydrogenation was performed according to Example 2 except that the alcohol was ethanol and the hydrogen pressure inside the reactor was 10 bar.

### Example 19

Furfural hydrogenation was performed according to Example 2 except that the alcohol was ethanol and the hydrogen pressure inside the reactor was 20 bar.

### Example 20

Furfural hydrogenation was performed according to Example 2 except that the alcohol was ethanol and the hydrogen pressure inside the reactor was 30 bar.

### Example 21

Furfural hydrogenation was performed according to Example 2 except that the alcohol was n-butanol.

### Example 22

Furfural hydrogenation was performed according to Example 2 except that the alcohol was n-butanol and the hydrogen pressure in the reactor was 10 bar.

### Example 23

Furfural hydrogenation was performed according to Example 2 except that the alcohol was n-butanol and the hydrogen pressure in the reactor was 20 bar.

### Example 24

Furfural hydrogenation was performed according to Example 2 except that the alcohol was n-butanol and the hydrogen pressure in the reactor was 30 bar.

The FeCo@GC of the present disclosure has numerous advantageous properties. The preparation strategy of the present disclosure offers a facile, effective, controllable, scalable process. Surprisingly, use of Co-MOF-71 as a Co source produced an efficient and selective FeCo catalyst because Co-MOF-71 is known to have poor electrical conductivity and low redox reactivity. However, the reduction of Fe³⁺ to Fe⁰ is facilitated by the Co atoms in Co-MOF-71 being surrounded by carbon atoms. Therefore, the use of a single-source for both Co and C in the form of Co-MOF-71 is important to producing the catalyst of the present disclosure. Furthermore, the use of Co-MOF-71 as the single source of Co and C produces a graphitic carbon shell that protects the FeCo core without the need for an additional shell-growing step.

Alloy chemistry was exploited to use FeCo@GC to efficiently catalyze selective hydrogenation of the furfural to produce levulinic acid (LA) with ethanol or the respective ether with IPA on demand. The hydrogenation of C═O occurred on the surface of Co, assisted by Fe, and protected by GC of the FeCo@GC system. High selectivity for the respective ether in IPA was achieved by forming a solvation shell of highly polar IPA around the catalyst's active site. The carbonyl functional group, rather than the five-membered ring, was hydrogenated on the cobalt surface, which was assisted by Fe and protected by the GC of the FeCo@GC bimetallic nanocomposite. By initiating hydrogenation at the carbonyl group instead of the five-membered ring, there is a synergistic effect that results in the high efficiency and selectivity exceeding 99%.

The magnetic FeCo@GC catalyst shows excellent chemical stability and high resistance to poisoning by chemicals present in the highly active complexing agent. The FeCo@C catalyst maintained high conversions efficiency and product specificity for four use cycles. Very high magnetic susceptibility data were recorded for FeCo@GC, indicating that the FeCo@C catalyst can be easily recovered by traditional magnetic materials.

A facile protocol for preparing a magnetic bimetallic nanocomposite, which might control the selectivity and activity of furfural hydrogenation over a Co-based catalyst is described. The method may also be used to design and form bimetallic catalysts for hydrogenation reactions with various substrates containing competitive functional groups.

Numerous modifications and variations of the present disclosure are possible in light of the above teachings. It is therefore to be understood that within the scope of the appended claims, the invention may be practiced otherwise than as specifically described herein.

1. A magnetically recoverable catalyst comprising:
a core material and a shell material;
wherein the core material is an Fe/Co alloy comprising Fe and Co in an equal molar ratio; and
wherein the shell material is graphitic carbon.

2. The magnetically recoverable catalyst according to claim 1, wherein the Fe—Co core is 3 to 4 nanometers in diameter.

3. The magnetically recoverable catalyst according to claim 1, having a particle diameter ranging from 20 to 250 nm.

4. The magnetically recoverable catalyst according to claim 1, having a particle diameter of about 40 nm.

5. The magnetically recoverable catalyst according to claim 1, wherein the core material has a body-centered cubic crystal structure.

6. The magnetically recoverable catalyst according to claim 1, wherein the core material has a Raman D-band to a G-band ratio (I_{D}/I_{G}) between 0.50 and 0.80 at the D-band equal to 1350-1370 and the G-band equal to 1575-1595 cm⁻¹.

7. A process for producing a magnetically recoverable catalyst comprising:
mixing nanoparticles of Fe₃O₄ with a Co metal-organic-framework material and grinding to form a precursor mixture, wherein the Fe and the Co are present in the precursor mixture in an equal molar ratio; and
heating the precursor mixture to at least 600° C. under an inert gas to form the magnetically recoverable catalyst.

8. The process for producing a magnetically recoverable catalyst according to claim 7, wherein the precursor mixture has an Fe₃O₄ nanoparticles to MOF-71 mass ratio of about 1:1.

9. The process for producing a magnetically recoverable catalyst according to claim 7, wherein a Fe₃O₄/MOF composite is the only compound present during the pyrolysis step.

10. The process for producing a magnetically recoverable catalyst according to claim 7, wherein the Co metal-organic-framework material mixed with the Fe₃O₄ nanoparticles is MOF-71 (Co).

11. The process for producing a magnetically recoverable catalyst according to claim 7, wherein the mixing includes dispersing the Fe₃O₄ nanoparticles having a diameter less than 5 nm in the Co metal-organic-framework material.

12. The process for producing a magnetically recoverable catalyst according to claim 7, wherein the heating includes heating to a temperature of from 600 to less than 900° C.

13. The process for producing a magnetically recoverable catalyst according to claim 7, wherein the heating includes heating to a temperature of from 600 to about 800° C.

14. A process for the selective hydrogenation of furfural comprising:
combining, in a reactor, furfural with a catalyst material comprising an Fe—Co alloy core and a graphitic carbon shell, wherein the Fe and Co are present in equal molar ratios;
adding an alcohol;
flushing the reactor at least one time with hydrogen gas;
pressurizing the reactor to a pressure of at least 10 bar with hydrogen gas; and
heating the reactor to a temperature of at least 140° C. to hydrogenate the furfural in the reactor in the presence of the catalyst material.

15. The process for the selective hydrogenation of furfural according to claim 14, wherein heating the reactor includes heating to a temperature of from 140° C. to 200° C.

16. The process for the selective hydrogenation of furfural according to claim 14, wherein the reactor is heated to about 170° C.

17. The process for the selective hydrogenation of furfural according to claim 14, wherein the reactor is pressurized to 40 bar during the heating.

18. The process for the selective hydrogenation of furfural according to claim 14, wherein the pressurizing and heating is conducted for about 20 hours.

19. The process for the selective hydrogenation of furfural according to claim 14, wherein the alcohol is a branched alcohol.

20. The process for the selective hydrogenation of furfural according to claim 14, wherein the alcohol is a straight chained alcohol.
